(19) United States
(12) Patent Application Publication
(10) Pub. No.: US 2022/0265894 A1
(43) Pub. Date: Aug. 25, 2022

(54) Antibiofilm Compositions, Wound Dressings, Cleaning Methods And Treatment Methods

(71) Applicant: Zoono Group Ltd, Botany South (NZ)

(72) Inventor: Paul Hyslop, Remuera (NZ)

(73) Assignee: Zoono Group Ltd, Botany South (NL)

(21) Appl. No.: 17/698,942

(22) Filed: Mar. 18, 2022

Related U.S. Application Data
(63) Continuation-in-part of application No. 17/180,161, filed on Feb. 19, 2021.

(30) Foreign Application Priority Data
Sep. 3, 2021 (EP) EP21194867.4

(51) Int. Cl. A61L 26/00; A61L 2/18

(52) U.S. Cl. CPC A61L 26/0066; A61L 26/0076; A61L 2/18; A61L 2300/404; A61L 2300/208; A61L 2300/216

(57) ABSTRACT

The patent disclosure covers antibiofilm compositions, wound dressings, cleaning methods and treatment methods. The antibiofilm composition is comprised of between about 0.05% (w/w) to about 0.20% (w/w) benzalkonium chloride; between about 0.20% (w/w) to about 0.50% (w/w) 3-(tri-methoxysilyl)propyldimethyl octadecyl ammonium chloride; between about 0.25% (w/w) to about 0.75% (w/w) phenoxyethanol and between about 98.55% (w/w) to about 99.5% (w/w) deionized water. Disclosed are wound dressings having a reservoir of the antibiofilm composition. In the field of inanimate surfaces, disclosed are methods to clean and clean and protect inanimate surfaces. In the field of animate surfaces, disclosed are methods for the treatment of nose, ear and skin/facial disorders associated with biofilms.

### CROSS-REFERENCE TO RELATED APPLICATIONS

This application claims priority from U.S. Non-Provisional application Ser. No. 17/180,161 which was filed on Feb. 19, 2021. The entirety of U.S. Non-Provisional application Ser. No. 17/180,161 which was filed on Feb. 19, 2021 is incorporated herein by reference.

This application also claims priority from PCT/IB2021/053643 which was filed on Apr. 30, 2021. The entirety of PCT/IB2021/053643 which was filed on Apr. 30, 2021 is incorporated herein by reference.

This application also claims priority from EP21194867.4 which was filed on Sep. 3, 2021. The entirety of EP21194867.4 which was filed on Sep. 3, 2021 is incorporated herein by reference.

### BACKGROUND OF THE INVENTION

### 1. Field of the Invention

This invention pertains generally to compositions with antibiofilm activity and more particularly to organosilane containing compositions for disrupting biofilm colonization on inanimate surfaces and animate wound surfaces.

### 2. Related Art

Inanimate surfaces are an ubiquitous part of human existence; for examples, there are surfaces associated with shelving displays in stores, tables in homes and floors in offices. Surfaces are present in both the medical environment (e.g., a hospital and doctor's office) and non-medical environment (e.g., home and office.)

Inanimate Surfaces are susceptible to the growth of biofilms (see, below.) Particularly vulnerable to the growth of biofilms are surfaces which are dirty, moist, wet and/or are in a medical environment. Biofilms on surfaces are injurious to human health and destructive to property.

Animate surfaces are an ubiquitous part of human existence; in particular, epithelium. Epithelium is a thin, continuous protective layer of cells which lines the outer surfaces of organs and inner surfaces of cavities of organs. The largest epithelium in a human body is the epidermis of skin covering almost an entire body. Other epithelium are sinus mucosal inside a nose and a canal inside an ear. (see, https://en.wikipedia.org/wiki/Epithelium.)

Epithelium, as well as other tissue, are vulnerable to infection by biofilms (see, below.) Amongst others, biofilm infections manifest themselves in the following medical conditions (A) disorders in the nose called sinusitis and chronic rhinosinusitis; (B) a disorder in the ear called chronic suppurative otitis (C) wounds characterized by lacerated and/or deleteriously-altered skin and/or other tissue occasioned by trauma; (D) wounds characterized by lacerated and/or deleteriously-altered skin and/or other tissue occasioned by certain diseases which do not allow soars to heal (e.g., diabetes); (E) burn injury occasioned by heat, radiation, corrosive chemical and/or electricity which deleteriously altered skin and/or other tissue, especially second and third degree burns characterized by extensive skin and/or other tissue alteration and (E) acne disfiguration occasioned by oil build-up in blocked skin follicles causing unsightly pimples.

A biofilm is a complex cluster of microbial cells that adhere to a surface (e.g., the sinus mucosal epithelium.) These clusters are embedded in a layer of extracellular polymers called the extracellular matrix. This extracellular matrix is produced by the bacteria when they are clustered in high numbers. The extracellular matrix provides protection to bacteria from the external environment, including being a physical barrier that impedes antibiotic penetration.

Setting aside that the extracellular matrix impedes the passage of antibiotics, antibiotics typically target metabolic processes of bacteria during high cell turnover. Bacteria living as biofilm undergo phenotypical and behavioral changes that often manifest in a reduced metabolic activity. Accordingly, antibiotics generally are ineffective against bacteria in biofilms which have reduced metabolic activity. Notwithstanding the reduced susceptibility to antibiotics, there are established clinical protocols for treating a biofilm infection with an antibiotic. In so attempting to treat a biofilm infection with an antibiotic, there is the challenging, costly and time-consuming task of first characterizing the bacteria in the biofilm in hopes of selecting a suitable antibiotic which will have activity against the bacteria in the biofilm.

All told, bacteria in biofilms are difficult to treat with systemic antibiotics because the bacteria in this state have a resistance to antibiotics up to one thousand times greater than planktonic forms of the same strain. Topical antibiotics fair no better with there being little data to support efficacy. Further, treatment with systemic antibiotics bears the risk of the development of antibiotic resistance bacteria. In treatment with topical antibiotics, there is a significant risk of development of antibiotic resistance bacteria with this route of administration.

As a broad and general proposition, known in the art is that quaternary ammonium compounds, such as the benzalkonium chloride and 3-(trimethoxysilyl)-propyldimethyloctadecyl ammonium chloride, can have effectiveness to eradicate biofilms and yet are safe to apply to human mucosa (see, Yuen, John, et al, Medical Implications of Antimicrobial Coating Polymers-Organosilicon Quaternary Ammonium Chloride, Modern Chemistry & Applications, Volume 1, Issue 3, 1000107 (2013).)

Notwithstanding, the mere knowledge that quaternary ammonium compounds have effectiveness is deficient (not good enough) to formulate a composition which provides real-world utility to treat a biofilm infected surface. The art of formulating quaternary ammonium compound compositions having real world effectiveness in eradicating biofilms is poorly understood and unpredictable. That is, not enough is known in the art to at theoretical level evaluate the potential of a composition to effectively eradicate bacterial biofilms due to various variables such as ability to penetrate the extracellular matrix of biofilms and to kill bacterial cells within the bacterial clusters.

Shelf life is the length of time that a commodity may be stored without becoming unfit for use. Shelf life can be influenced by contamination by micro-organisms. Water based products attract bacteria. (see, https://en.wikipedia.org/wiki/Shelf life.) Shelf life significantly affects the commercial viability of a product, including anti-biofilm compositions. As a general proposition, longer shelf life is sought by manufacturers, fulfillment houses, distributors, retailers and others in the supply chain, medical professional intermediaries, other professional intermediaries and end users.

Patent publication WO 2015/139085 A1 by Jeffrey Paul Smyth et al. entitled Antimicrobial Sanitizer Compositions and Their Use having a publication date of Sep. 24, 2015 discloses a sanitizing composition for use on skin (i.e., hands and feet) and for air sanitization. The sanitizing composition is broadly comprised of (i) an alkoxy silyl ammonium film-forming compound; (ii) a benzalkonium or benzethonium chloride; (iii) a polymeric biguanide and (iv) a surfactant system comprising an alcohol ethoxylate and (v) an alkylglucoside or alkylpolyglycoside. In some embodiments, the compositions are aqueous compositions wherein the carrier comprises water.

In particular embodiments of Smyth et al's composition, the alkoxy silyl ammonium film-forming compound is 3-triethoxysilylpropyl-N,N-dimethyl-N-octadecyl ammonium chloride. It is present in a range of 0.1% to 1.5% w/w, especially about 0.3% to 1.0% w/w with a specific call out of range from about 0.4 to 0.6% w/w of the composition. Smyth el al. report that alkoxy silyl quaternary ammonium compounds hydrolyzes in water forming the trihydroxy silicon functional group.

In some embodiments, the four constituency on the quaternary nitrogen of benzalkonium are benzyl, R₂, R₃ and R₄ where R₂ and R₃ are independently selected from C₁ to C₆ alkyl and R₄ is C₈-C₁₈ alkyl or —(CH₂CH₂O)₂[4-(1-dimethyl-3-dimethylbutyl)phenyl. The benzalkonium chloride is present in an amount ranging from 0.1% to 1.0% w/w of the composition, especially about 0.1% to about 0.5% w/w, more especially about 0.1% to 0.2% w/w of the composition, and even more especially about 0.1% to 0.13% w/w of the composition.

Smyth et al. conducted microbial suspension tests and plating tests to validate effectiveness against bacteria in general. They report that their compositions are effective for immediate sanitizing and providing a persisting protective effect 3, 6 or 12 hours with speculation about some level of protective effect beyond 12 hours. There is no testing related to biofilms and the composition is not touted as effective against biofilms.

Smyth et al. denigrate alcohol-based hand sanitizers on the grounds of a loss of effectiveness after the alcohol evaporates and that continued use of alcohol-based skin sanitizers causes skin damage. Smyth et al. make no mention of an alcoholic additive as a preservative when the composition is stored in a bottle prior to alkoxy silyl ammonium film-forming and/or that film formation prior to application to a surface is undesirable.

US Patent Publication 2015/0328241 A1 by Carl Hilliard et al entitled Product and Method for Treatment of a Biofilm Including Control of Substrate Colonization and Treatment Infection having a publication date of Nov. 19, 2015 discloses an antimicrobial product, inclusive of microbes in a biofilm, having as an active ingredient a quaternary ammonium compound. Included amongst the quaternary ammonium compounds that can be formulated into the product is the use of 3-(trimethoxysilyl) propyl dimethyl octadecyl ammonium molecule.

The publication teaches a broad concentration range for organosilane in formulating product from less than 0.10% by weight to greater than 5.0%. Hilliard et al provide guidelines for selecting the concentration of organosilane; namely, (i) in situations of established biofilms, higher concentrations for stronger electrostatic attractive forces and detergent effects resulting in higher antimicrobial activity; (ii) in situations of exfoliation and dressing changes, higher concentration up to and over 5% by weight to allow the product to act as an antiseptic for a longer period of time with a note that concentrations over 3% increase polymerization of the organosilane within the stored product prior to use and (iii) in situations of cutaneous epithelium and open wounds where product shedding occurs, lower concentrations of organosilane down to and below 0.1%, so as to lower the amount of organosilane discharged into the environment and thereby mitigate against natural selection of microbes resistant to organosilane (unestablished is that such natural occurs and if so, to a level endowing a bacteria with immortality to the organosilane—perhaps, at most, a slowing of the kill rate.) There is a claim specifically directed at the range of between 0.1% and 1.00%.

Hilliard et al teaches having a carrier as a component of their product. The carrier performs the function of an excipient. One class of carrier compound taught is an alcohol and they provide one, and only one, specific example of an alcohol; namely, cetyl alcohol. No concentration for the carrier alcohol is disclosed. There is no mention of the carrier also functioning as a preservative. Hilliard et al further teaches having an antiseptic as component of their product. They disclose a lengthy list of antiseptics and included within this list is benzalkonium chloride and ethyl alcohol. No other alcohols are identified. No concentrations of antiseptics are disclosed.

Chinese patent publication CN107412213 by Likai Fu entitled Multifunctional Antibacterial and Sterilizing Drug for External Use and Preparation Method Thereof having a publication date of Dec. 1, 2017 discloses an antibacterial sterilizing agent comprised of in parts by weight of 0.05-0.2% benzalkonium chloride, 0.05-2% organosilicon quaternary ammonium salt where the organosilicon quaternary ammonium salt is [3-(trihydroxy silicon base) propyl] dimethyl octadecyl ammonium chloride and 97.8 to 99.9% deionized water. This publication more specifically teaches a composition having in parts by weight 0.1% benzalkonium chloride; 0.2% organosilicon quaternary ammonium salt where the organosilicon quaternary ammonium salt is [3-(trihydroxy silicon base) propyl] dimethyl octadecyl ammonium chloride salt and 99.7% deionized water.

Chinese patent publication CN107412213 expresses an unequivocal disdain for, and in clear terms disparages, alcohol as part of the composition. In particular, Fu states as an advantage of his composition that “[t]he bactericide adopts deionized water, does not contain alcohol, and increases the safety of the product.” He goes on further to write that “[t]he preparation has the advantages of safety, no toxicity, no alcohol, etc.” Moreover, in a “cut and paste” fashion Fu repeats and reiterates on a plurality of occasions the foregoing statements in his publication and thereby forcefully teaches away from alcohol. Fu's teaching completely discourages anyone in the art from contemplating enhancing shelf life with an alcoholic preservative.

Chinese Patent CN110897914 A by Gouzhong You et al entitled Foam Type No-clean Antibiofilm and Preparation Method Thereof having publication date of Mar. 24, 2020 discloses a sanitizer for use in the absence of running water for meeting sanitary standards. The sanitizer is comprised of by parts per weight of 0.1 to 1.0 parts decyl glucoside, 0.4 to 4.0 parts foaming agent, 0.01 to 0.1 parts benzalkonium chloride, 0.5 to 5.0 parts glycerol, 0.1 to 1.0 parts polysorbate, 0.01 to 0.1 parts witch hazel extract, 0.01 to 0.1 parts aloe extract, 0.2 to 1.0 part phenoxyethanol, 0.01 to 0.5 parts essence and the balance deionized water to 100.0 parts. The composition is not touted for having antibiofilm activity and/or for its shelf life.

European Patent Application EP 3 662 884 A1 by Theodoros Lanaras entitled Wet and Refreshing Wipe having a publication date of Jun. 10, 2020 discloses a cleansing solution for impregnating an artificial fabric with the ultimate production of a wet and refreshing wipe. The cleansing solution is comprised of 99.10-99.50% water; 0.25-0.30% phenoxyethanol; 0.18-0.22% fragrance; 0.10-0.14% ethylhexylglycerin and 0.08-0.12% benzalkonium chloride. Lanaras teaches that the cleansing solution be greater than 99% water so as allow the wipe to be used in any area of the body and with a very high moistening and refreshing capacity. Conversely, to maintain 99+% water, Lanaras teaches away from higher concentrations phenoxyethanol and/or adding other active ingredients to the cleansing solution. The composition is not touted for having antibiofilm activity.

Australian patent application publication number AU 20211200502 B1 by Hartly Campbell Atkinson entitled A Disinfectant having a publication date of May 27, 2021 discloses a human skin disinfectant utilizable, inter alia, for hands. In broad terms, the disinfectant is comprised of 0.2%-0.4% wt organosilane and 0.08%-0.12% wt benzalkonium, where the organosilane can be 3-(trimethoxysilyl)propyl dimethyl octadecyl ammonium salt. Campbell teaches to add phenoxyethanol as a preservative in a broadest range of 0.7%-1.3% wt.

Campbell purports to validate his disinfectant for effectiveness against bacterial and virus suspensions with a residual effectiveness measured after 24 hours. The disinfectant is not touted for being effective against bacteria in a biofilm. The disinfectant is not touted for having a residual effectiveness longer than 24 hours. The disinfectant is not touted for having extended multiyear shelf life.

A deficiency of compositions in the art is effectiveness to penetrate the extra-cellular matrix of a biofilm so as to kill bacteria in a biofilm and otherwise eradicate and eliminate a biofilm.

A deficiency of compositions in the art is effectiveness to reduce and inhibit bacteria in a biofilm so as to achieve a degree needed to control the biofilm.

A deficiency in the art is a means to treat a biofilm infected animate surface without having to first characterize the bacteria in the biofilm.

A deficiency in the art is to treat a biofilm infected surface without promoting the natural selection of antibiotic resistant bacteria.

A deficiency in the art is the shortness of the shelf life of compositions to eradicate biofilms and more particular, shelf lives which are less than three to about four years.

Accordingly, there exists a need for an improved formulation of a composition to penetrate the extra-cellular matrix of a biofilm, to kill bacteria in a biofilm and otherwise eradicate and eliminate a biofilm.

There exists a need for an improved formulation of a composition to reduce and inhibit bacteria in a biofilm so as to achieve a degree needed to control the biofilm.

There exists need for a means for controlling or eradicating bacteria in a biofilm infected animate surface without having to characterize the bacteria in the biofilm.

There exists a need for a non-antibiotic agent that effectively eradicates bacteria in a biofilm while maintaining safety to apply to human epithelial.

More specifically, there exists a need for a quaternary ammonium compound type antibiofilm agent with improved ability to penetrate the extracellular matrix of biofilms and to kill the bacterial cells within the bacterial clusters.

There exists a need for an improved formulation of an antibiofilm composition having a longer shelf life and more particularly, a shelf life of at least about three years.

The present invention has properties that suggest it is likely to be such an agent to satisfy these needs. In contrast to common antibiotics, the present invention exerts its activity via a combined physicochemical mode of action (rather than metabolic actions.) The physical properties of the present invention, inter alia, rupture the microbial cell wall/membrane, and thereby kill the organism. The chemical properties, inter alia, prevent additional replication of remaining bacterial cells. Additionally, the present invention is extremely well tolerated on human mucosal surfaces.

The present invention satisfies the aforementioned needs, as well as others, and generally overcomes the presently known deficiencies in the art.

### SUMMARY OF THE INVENTION

The present invention is directed to antibiofilm compositions. The invention has, inter alia, two overarching fields of use. One overarching field of use is as an inanimate surface disinfectant and protectant. The other overarching field of use is medical. The medical field of use subdivides into two main subfields of use. One main subfield of use is as a therapeutic delivered in liquid form for treatment of nose, ear, skin/facial and other tissue disorders associated with biofilms. The other main subfield of use is wound dressings, bandages and other devices having a reservoir of the antibiofilm composition.

An object of the present invention is an improved formulation of a composition to penetrate the extra-cellular matrix of a biofilm, kill bacteria in a biofilm and otherwise eradicate and eliminate a biofilm.

An object of the present invention is an improved formulation of a composition to reduce and inhibit bacteria in a biofilm so as to achieve a degree needed to control the biofilm.

An object of the present invention is an improved formulation of a non-antibiotic composition for effectively eradicating biofilms while maintaining safety to apply to human mucosa and concomitantly, not having a selective evolutionary pressure generating super microbes resistant to being killed.

An object of the present invention is treatment protocols for controlling or eradicating bacteria in biofilm infected animate surface (e.g., epithelial.)

A further object of the present invention is treatment protocols for controlling or eradicating bacteria in biofilm infected animate surface without having to first characterize the bacteria in the biofilm, albeit that a kill rate varies with species of bacteria.

A more specific object of the present invention is an improved formulation of a quaternary ammonium compound type antibiofilm agent with improved ability to penetrate the extracellular matrix of biofilms and to kill bacterial cells within the bacterial clusters.

An object of the present invention is an improved formulation of an antibiofilm composition having a shelf life longer than that of formulations in the prior art including at least about three years and boding to about four years.

One aspect of the present invention is an antibiofilm composition comprised of (a) between about 0.05% (w/w) to about 0.20% (w/w) benzalkonium chloride; (b) between about 0.20% (w/w) to about 0.50% (w/w) 3-(tri-methoxysilyl)propyldimethyl octadecyl ammonium chloride; (c) between about 0.25% (w/w) to about 0.75% (w/w) phenoxyethanol and (d) between about 98.55% (w/w) to about 99.5% (w/w) deionized water.

Another aspect of the present invention is a wound dressing comprised of (i) a bandage component and (i) a pad component that has as a reservoir capability which is impregnated with the above described antibiofilm composition. The antibiofilm composition in the reservoir is communicable to epithelium and/or other tissue having a biofilm associated infirmity.

Another aspect of the present invention is a method for cleaning an inanimate surface having on it a biofilm. The method is comprised of a plurality of steps. The first step is applying to the inanimate surface the above described antibiofilm composition. A further step is allowing the benzalkonium chloride to penetrate the biofilm extracellular matrix and kill bacteria. A further step is allowing the 3-(tri-methoxysilyl)propyldimethyl octadecyl ammonium chloride to penetrate the biofilm extracellular matrix and kill bacteria. A further step is allowing the 3-(tri-methoxysilyl)propyldimethyl octadecyl ammonium chloride to dry on the inanimate surface with the covalent bonding of the 3-(tri-methoxysilyl)propyldimethyl octadecyl ammonium chloride to form a positively charged micro thin coating on the surface. This micro thin coating electrostatically clings negatively charged bacteria, pierces their outer membrane or coat and kills the microorganism. Accordingly, for an extended period of between at least about 1 day to at least about 30 days bacteria are prevented from forming a biofilm.

Another aspect of the present invention is a prophylactic method against the formation of a biofilm on an inanimate surface. The method is comprised of a plurality of steps. A first step is applying to the inanimate surface the above described antibiofilm composition. A further step is allowing the 3-(tri-methoxysilyl)propyldimethyl octadecyl ammonium chloride to dry on the inanimate surface with the covalent bonding of the 3-(tri-methoxysilyl)propyldimethyl octadecyl ammonium chloride to form a positively charged micro thin coating. This charged micro thin coating functions as described above. Accordingly, for an extended period of between at least about 1 day to at least about 30 days, bacteria are prevented from forming a biofilm.

Another aspect of the present invention is a method of treating a living subject having an epithelium and/or other tissue that is infected by a biofilm associated infirmity. The method is comprised of a plurality of steps. A first step is accommodating for treatment a living subject having epithelium and/or other tissue that has biofilm associated infirmity. A further step is ascertaining the location of the epithelium and/or other tissue that has the biofilm associated infirmity and that said location is assessable. There is no need to characterize the bacteria in the biofilm. A further step is applying to the located epithelium and/or other tissue the antibiofilm composition described above until the infection subsides.

The previously described versions of the present invention have many advantages which include an ability to effectively penetrate the extra-cellular matrix of a biofilm and after so penetrating being potent to kill bacteria; being extremely well tolerated on human mucosal surfaces; killing by a physicochemical mode of action of rupturing bacterial outer membranes or cell walls which substantially surpasses the ability of bacteria to mutate into resistant strains so as not to contribute to the evolution of super microbes that cannot be killed; for the same reason, a very broad range to effectively near universal kill capability of bacteria in a biofilm and eliminating the need for characterization of the bacteria in the film, albeit that a kill rate varies with species of bacteria; a kill effectiveness at a level that controls or eradicates a biofilm; a long mode of action preventing additional replication of microbes remaining after the initial kill into a biofilm and enhanced commercial viability from a longer shelf life than compositions in the prior art, including a shelf life of at least about three years boding to about four-years.

### BRIEF DESCRIPTION OF THE DRAWINGS

These and other features, aspects and advantages of the present invention will become better understood with reference to the following description, appended claims and accompanying drawings where:

FIG. 1A shows a planar view of the setup for a CDC Biofilm Reactor by Biosurface Technologies Corporation (Bozeman, Mont., USA) (CDC-BR), along with a blow-up box delineating a reactor vessel, as background for the colonization and testing in Examples 3 and 4 of the efficacy of the present invention;

FIG. 1B shows a breakaway perspective view of the CDC-BR reactor vessel in FIG. 1A delineated by the blow-up box, as background for the colonization and testing in Examples 3 and 4 of the efficacy of the present invention;

FIG. 2 shows a schematic view of the workflow utilizing the CDC-BR of FIGS. 1A and 1B for antibiofilm treatment and bacterial enumeration in determining biofilm eradication at certain concentrations in testing in Example 4 of the efficacy of the present invention;

FIG. 3A is tabular showing of data on colony forming units (CFUs) recovered after treating Staphylococcus aureus American type culture collection (ATCC) 6538 (S. aureus) biofilms at certain concentrations from testing in Example 4 of the efficacy of the present invention;

FIG. 3B is a graphical showing summarizing the results of the time-kill experiments on S. aureus ATCC 6538 at certain concentrations from testing in Example 4 of the efficacy of the present invention;

FIG. 4A is a tabular showing of data on CFUs recovered after treating Pseudomonas aeruginosa (P. aeruginosa) ATCC 27586 biofilms at certain concentrations from testing in Example 4 of the efficacy of the present invention;

FIG. 4B is a graphical showing summarizing the results of the time-kill experiments on P. aeruginosa ATCC 27586 biofilms at certain concentrations from testing in Example 4 of the efficacy of the present invention;

FIG. 5A shows a perspective view of a human ear having an outer ear, an ear canal opening and an ear canal with a biofilm infection in the ear canal, along with cross section arrows, as background for the present invention;

FIG. 5B shows a planar cross-sectional view of the infected ear in FIG. 5A along the indicated cross-sectional arrows where there is an antibiofilm composition dispensing dropper above and adjacent to the ear canal opening in the outer ear in accordance with a treatment regimen pursuant to the present invention;

FIG. 5C shows the planar cross-sectional view of FIG. 5B where the antibiofilm composition has been dispensed and resides on the biofilm infection in the ear canal in accordance with a treatment regimen pursuant to the present invention;

FIG. 5D shows the planar cross-sectional view of FIG. 5C at a time later where the biofilm infection in the ear canal is subsided;

FIG. 6A shows a perspective view of a human with an arm wound that is biofilm infected and a nurse holding a bandage in accordance with the present invention in proximity to the wound, along with a blow-up box delineating a portion of the bandage, and further illustrating the start of a treatment regimen pursuant to the present invention;

FIG. 6B shows a planar cross-sectional view of the portion of the bandage in FIG. 6A delineated by the blow-up box where there is an antibiofilm composition impregnated pad according to the present invention;

FIG. 6C is a breakaway perspective view of the bandage in FIG. 6B bandaging the wound in FIG. 6A in accordance with a treatment regimen pursuant to the present invention;

FIG. 7A shows a perspective view of a floor having on it a biofilm, as background for the present invention and

FIG. 7B shows a perspective view of the floor in FIG. 7A where antibiofilm composition is being spray applied to the floor in accordance with the present invention.

### DETAILED DESCRIPTION OF THE INVENTION

The present invention is described more fully in the following disclosure. In this disclosure, there is a discussion of embodiments of the invention and references to the accompanying drawings in which embodiments of the invention are shown. These specific embodiments are provided so that this invention will be understood by those skilled in the art. This invention is not limited to the specific embodiments set forth herein below and in the drawings. The invention is embodied in many different forms and should be construed as such with reference to the appended claims.

As written above, the present invention is directed to antibiofilm compositions. The invention has, inter alia, two overarching fields of use. One overarching field of use is as an inanimate surface disinfectant and protectant. The other overarching field of use is medical. The medical field of use subdivides into two main subfields of use. One main subfield of use is as a therapeutic delivered in liquid form for treatment of nose, ear, skin/facial and other tissue disorders associated with biofilms. The other main subfield of use is wound dressings, bandages and other devices having a reservoir of the antibiofilm composition.

In general terms and for an overview, embodiments of the anti-biofilm composition are comprised of the following major components: benzalkonium chloride, quaternary silane 3-methoxysilyl)propyldimethyl octadecyl ammonium chloride, phenoxyethanol and deionized water. Optionally, there is a fragrance. In the discussion that follows, each of these major components is discussed, along with other components/structures in the embodiments of this invention. Thereafter, there is a discussion on how to make and use the antibiofilm composition and various other embodiments of the invention.

Benzalkonium chloride (BZK) is a microbiocidal agent in the family of quaternary ammonium compound. The molecule has the chemical structure shown below:

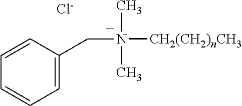

where n=8, 10, 12, 16 and 18. The molecule has IUPAC names of N-Alkyl-N-benzyl-N,N-dimethylammonium chloride and Alkyldimethylbenzylammonium chloride.

Preferably, benzalkonium chloride is that of the same which meets the description of Chemical Abstracts Service No. 68424-85-1. That is, quaternary ammonium compounds, benzyl-C12-16-alkyldimethyl, chlorides. As of the filing of the application for this patent, such benzalkonium chlorides are listed substances with the United States Environmental Protection Agency (EPA) and assigned tracking number 429654. Therefore, compounding with the same bodes well for getting regulatory approval and registration of a composition. More preferably, the alkyl component is C12 (dodecyl), C14 (Myristyl), a blend of 40% C12, 50% C14 and 10% C10.

As introduced above, benzalkonium chloride is a microbiocidal agent. Benzalkonium chloride is believed to have fast-acting capabilities. Benzalkonium chloride is thought to have both bactericidal and virucidal activity, as well as being active against fungi and protozoa. A postulated mode of action is that benzalkonium chloride is a cationic surfactant that dissociates a microbial cellular membrane and thereby compromising cellular permeability controls and inducing leakage of cellular contents. Also postulated is that benzalkonium chloride disrupts/dissociates intermolecular interactions and denatures the tertiary structure of proteins. The result being the deactivation of enzymes essential to microbe respiratory and metabolic activities.

The concentration of the benzalkonium chloride in the antibiofilm composition ranges from about 0.05% percent by weight (w/w) to about 0.20% percent by weight (w/w). In a more preferred embodiment, the concentration of the benzalkonium chloride is about 0.10%. The benzalkonium chloride at the above concentrations in combination with other ingredients at the concentrations, and relative concentrations, as taught herein below is believed to act in a synergistic manner with a disinfection that is heightened from that which can be expected based on the prior art.

The quaternary ammonium silane 3-(tri-methoxysilyl)propyldimethyl octadecyl ammonium chloride has the chemical structure shown below:

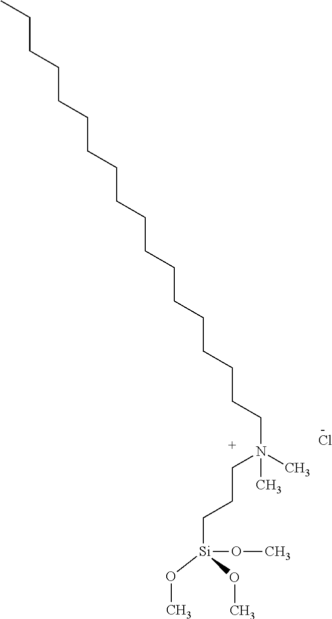

The molecule has the IUPAC name of 1-Octadecanaminium, N,N-dimethyl-N-[3-(trimethoxysilyl)propyl]-, chloride (1:1). The molecular formula is C26H58NO3Si.Cl and the molecular weight is 496.29.

Preferably, the quaternary ammonium silane 3-(tri-methoxysilyl)propyldimethyl octadecyl ammonium chloride is that of the same which meets the description of Chemical Abstracts Service No. 27668-52-6. As of the filing of the application for this patent, such quaternary ammonium silane 3-(tri-methoxysilyl)propyldimethyl octadecyl ammonium chloride is a listed substances with the United States Environmental Protection Agency (EPA) and assigned tracking number 246561. Therefore, compounding with the same bodes well for getting regulatory approval and registration of a antibiofilm composition.

The 3-(tri-methoxysilyl)propyldimethyl octadecyl ammonium chloride is microbiocidal and believed to have a long lasting capabilities. The primary mode of action of the 3-(tri-methoxysilyl)propyldimethyl octadecyl ammonium chloride is discussed immediately below. This discourse on the primary mode of action necessarily overlaps with a discourse on method of use the antibiofilm composition. Later in this disclosure, there is further discussion on method of use.

As a prefatory matter, there is no requirement or step for admixing 3-(tri-methoxysilyl)propyldimethyl octadecyl ammonium chloride and deionized water (discussed below); i.e., the composition is ready to use. The composition is applied to an inanimate or animate surface. The applying to an inanimate surface is done by misting, spraying, brushing or dipping. The composition is applied to an animate surface by dropping, flushing and/or wicking from a reservoir in a bandage.

When the composition dries, a condensation reaction occurs on the surface where there is the formation of a micro thin coating of polymerized monomers of 3-(tri-methoxysilyl)propyldimethyl octadecyl ammonium with strong covalent bonds between the monomers. The octadecyl alkyl side chain lineup pointing away from the surface. This can be likened to having millions of sword-shaped “road spikes” on the surface. At the base of each of these “swords” is a positively charged quaternary carbon unit.

Each of positively charged quaternary carbon units performs a dual function. One function is to attach the micro-coating to commonly found surfaces which bear a negative charge. The other is to attract/trap microbes with a negatively charged outer membrane. Typically, microbes have an outer membrane comprised of lipoproteins which have long chain fatty acids and glycerides that are anionic in nature.

The alkyl side chain then pierces the outer membrane of the attracted/trapped microbe with organism lysis. The microbe is killed via a form of electrostatic electrocution. The same monomer in the polymerized micro-coating is capable of attacking a new microbe again and again with only a single application. Because the killing of microorganisms is not by poisoning with toxic chemicals, but, rather, goes to the fundamental architecture of microorganisms, microorganisms have almost no capability, to no capability to adapt to it. Accordingly, there is almost no possibility, to no possibility, of a defensive mutation and consequently no possibility of superbugs forming. At best, a microorganism can evolve to slow down the kill rate.

In more detail, the 3-(tri-methoxysilyl)propyldimethyl octadecyl ammonium chloride first undergoes an hydrolysis in conjunction with the deionized water (discussed below) to enter into an equilibrium with 3-(tri-hydoxysilyl)propyldimethyl octadecyl ammonium chloride. This hydrolysis is depicted in the chemical drawing and chemical equation shown below:

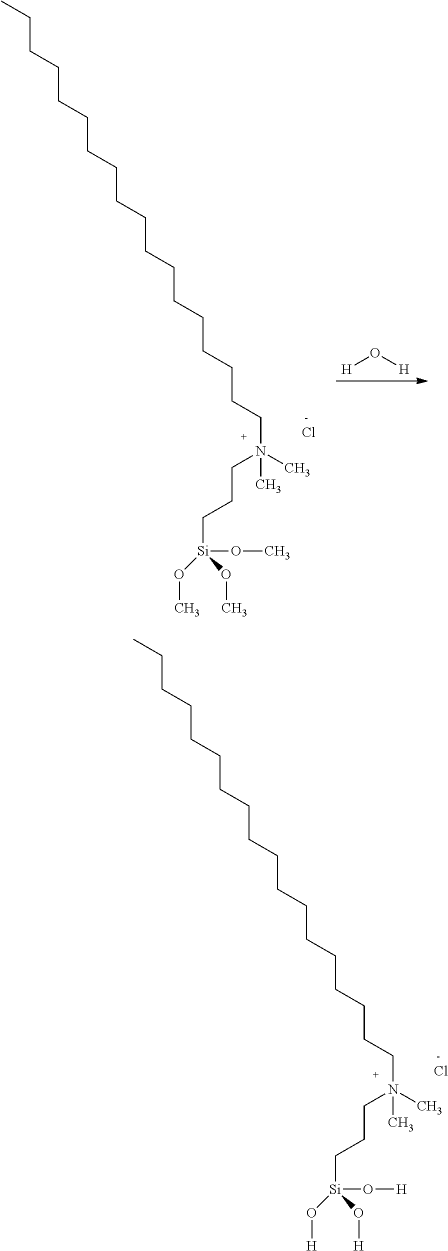

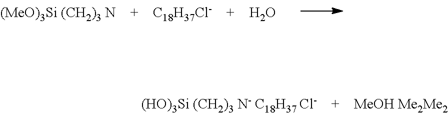

The condensation/polymerization reaction is depicted in the chemical drawing and chemical equation shown below:

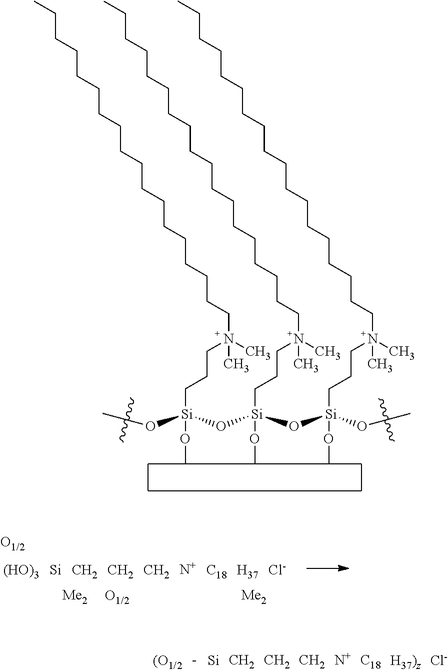

As a secondary mode of action, the 3-(tri-methoxysilyl)propyldimethyl octadecyl ammonium chloride is believed to have microbiocidal activity in unpolymerized fluid form. As a liquid phase agent not condensed or polymerized, the 3-(tri-methoxysilyl)propyldimethyl octadecyl ammonium chloride functions to kill microbes in a similar manner and fashion to that when it is condensed or polymerized into a micro-coating. That is, the positively charged quaternary carbon unit attracts/traps a microbes with a negatively charged outer membrane. The alkyl side chain then pierces the outer membrane of the attracted/trapped microbe with organism lysis. The microbe is killed via a form of electrostatic electrocution.

The concentration of the 3-(tri-methoxysilyl)propyldimethyl octadecyl ammonium chloride in the antibiofilm composition ranges from about 0.20% percent by weight (w/w) to about 0.50% percent by weight (w/w). In a more preferred embodiment, the concentration of 3-(tri-methoxysilyl)propyldimethyl octadecyl ammonium chloride is about 0.20%. The 3-(tri-methoxysilyl)propyldimethyl octadecyl ammonium chloride at the above concentrations in combination with the other ingredients at the concentrations, and relative concentrations, as taught herein is believed to act in a synergistic manner with disinfection that is heightened from that which can be expected based on the prior art.

Phenoxyethanol is a microbiocidal agent/preservative, emulsifier and fixative for a fragrance (discussed below). The chemical formula is C₈H₁₀O₂ or C8H10O2. The molecule has the chemical structure shown below:

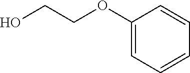

The molecular weight in grams per mole is 138.166. The molecule has the IUPAC name of 2-Phenoxyethanol. Preferably, the phenoxyethanol is that of the same which meets the description of Chemical Abstracts Service No. 122-99-6.

As introduced above, the phenoxyethanol is believed to perform the function of a microbiocidal agent and preservative. Phenoxyethanol is believed active against Gram negative bacteria, Gram positive bacteria and yeast. The phenoxyethanol is also believed to be an emulsifier effective against local phases forming within the composition and keeping all ingredients in solution. The phenoxyethanol is also believed to have activity as a fixative for a fragrance.

The concentration of phenoxyethanol in the antibiofilm composition ranges from about 0.25% percent by weight (w/w) to about 0.75% percent by weight (w/w). In a more preferred embodiment, the concentration of phenoxyethanol is about 0.50%. The phenoxyethanol at the above concentrations in combination with other ingredients at the concentrations, and relative concentrations, as taught herein is believed to act in a synergistic manner with disinfection that is heightened from that which can be expected based on the prior art.

Here is an aside with background information. The antibiofilm composition is water based (see, below.) A water-based product attracts bacteria. The antibiofilm composition may or may not be sterile packaged and hermetically sealed. Doing so increases product manufacturing expense and ultimate product cost and typical this is not done. Even if the composition is sterile and hermetically packed, the effectiveness sterile packaging and hermetical sealing erodes with time. Due note is made that (A) until applied to an inanimate or animate surface, the benzalkonium chloride and 3-(tri-methoxysilyl)propyldimethyl octadecyl ammonium chloride are not at full potency so as to effectively be a preservative when the composition is stored in liquid form.

Returning to the main line of disclosure, as mentioned above, the phenoxyethanol performs the function of being a preservative. As the water-based products attracts bacteria over a period of years, the phenoxyethanol at the above concentrations, acting alone and/or in combination with the other ingredients, knocks down the bacteria count and keeps the bacteria down to afford the antibiofilm composition an at least about three-year shelf life, with boding to four years, when bulked packed, bottled for individual use and/or absorbed into a reservoir of a bandage. An at least about three-year shelf life is a real-world advantage sought by manufacturers, fulfillment houses, distributors and retailers in the supply chain, medical professional intermediaries, other intermediaries and end users.

Deionized water is the base/solvent. Water is a molecule with an oxygen atom and two hydrogen atoms singling bounded to the oxygen atom. Water has the chemical formula H₂O or H2O. The molecule has the chemical structure shown below:

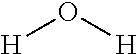

Water has a molecular weight in grams per mole of 18.01528. Being deionized, as a to-be-added ingredient to the composition, the concentration of the water approaches 100%. Accordingly, the melting point of deionized water approaches 0° C. (degrees Celsius) and the boiling point approaches 100° C. At 20° C., deionized water has a vapor pressure of 17.5 Torr. Preferably, the deionized water is that of the same which meets the description of Chemical Abstracts Service No. 7732-18-5.

In addition to being a base/solvent into which the other ingredients are dissolved, the deionized water is also reactant in the hydrolysis discussed above of 3-(tri-methoxysilyl)propyldimethyl octadecyl ammonium. The evaporation of the deionized water is integral with the condensation/polymerization of the 3-(tri-methoxysilyl)propyldimethyl octadecyl ammonium into a microcoating, as discussed above.

The concentration of deionized water in the antibiofilm composition ranges from about 98.55% percent by weight (w/w) to about 99.50% percent by weight (w/w). The particular concentration of deionized water is a function of the concentration of all other ingredients so that total of every ingredient, including deionized water, is 100%. Accordingly, targeted concentrations are set for benzalkonium chloride, 3-(tri-methoxysilyl)propyldimethyl octadecyl ammonium chloride and phenoxyethanol and deionized water being present in a concentration to achieve the concentrations set for the other ingredients. In a more preferred embodiment, where the benzalkonium chloride, 3-(tri-methoxysilyl)propyldimethyl octadecyl ammonium chloride and phenoxyethanol are at the above preferred concentrations and the preferred concentration of deionized water is therefore about 99.2% (w/w).

Optionally, and preferably for applications on inanimate surfaces, the antibiofilm composition includes a fragrance. Examples of suitable fragrances are bergamot, gardenia, honeysuckle, jasmine, lavender, pear, peony, rosemary, sandalwood and vanilla. The concentration of fragrance in the antibiofilm composition ranges from about 0.05% percent by weight (w/w) to about 0.15% percent by weight (w/w). In a more preferred embodiment, the concentration of fragrance is about 0.10%. The concentration of deionized water being proportionally less to achieve the foregoing targeted concentrations of fragrance.

The liquid antibiofilm composition is made/manufactured by mixing and blending of raw materials, supra, in concentrations as specified, using standard equipment for chemical mixing and blending and standard procedures for using the equipment. The mixed and blended raw materials are bottled and packaged using standard bottling and packaging equipment in accordance with standard procedures for that equipment. That is, the compositions are made using for examples, liquid storage containers, ingredient and product handlers, in-line mixers, co-rotating twin-screw mixers, homogenizers, bottling equipment, product packaging equipment and automation and electronic controls, all substantially in the manner and in accordance with that prescribed by the manufacturer(s) of the equipment. Preferably, the manufacturing facility is certified by the environmental protection agency or other regulatory agency having jurisdiction.

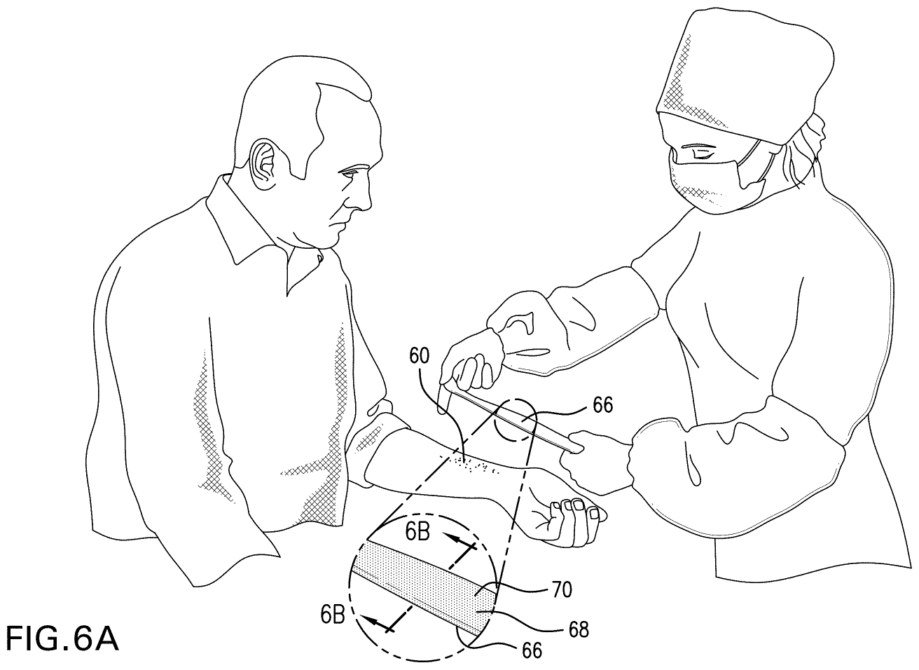

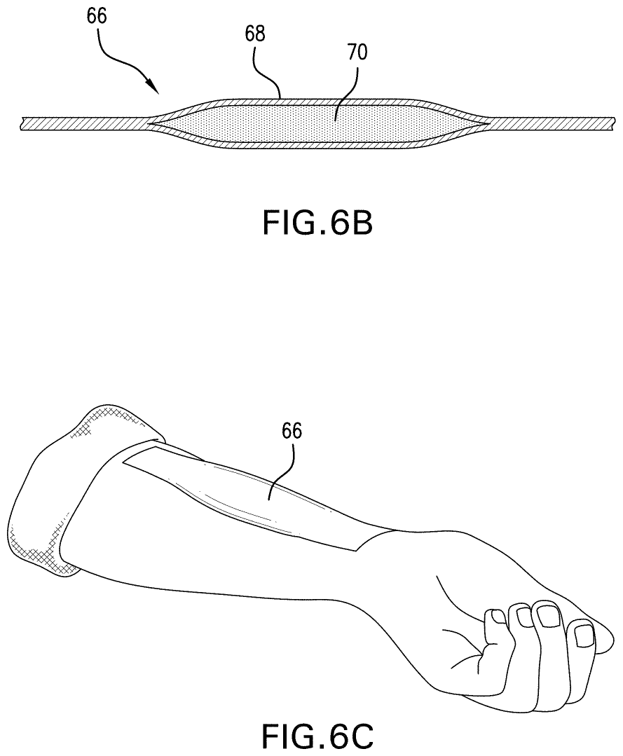

Referring to FIGS. 6A and 6B, embodiments of this invention are a wound dressing (66). Typically, the wound dressing is comprised of bandage component (65) and a pad or mat component (68). The pad component (68) functions as a reservoir which is impregnated with the antibiofilm composition according to the present invention. The antibiofilm composition in the reservoir is communicable by way of osmotic, hydrodynamic and physical forces to epithelium and/or other tissue having a biofilm associated infirmity (see, Example 7.)

The wound dressing (66) is made/manufactured using conventional materials, other than the antibiofilm composition, with conventional equipment and according to standard procedures for making medicated bandages. Briefly, materials, other than the antibiofilm composition, are adhesives, cotton fabrics, gauze, plastic banding, packaging material and/or silicone strips. The manufacturing is undertaking using cutting equipment, folding machines, hot air-drying ovens, packing equipment, pressing machines, rolling machines, steam sterilizers, trimming machines and winding equipment. For the purposes of enablement only, incorporated herein are U.S. Pat. No. 5,147,338 (Lang) for “Medicated, low adherency wound dressings” which is directed to the manufacture and use of absorptive wound dressings for treatment of burns and other wounds and U.S. Pat. No. 4,117,841 (Perrotta) for “Medicated bandage pocket” which is directed to the manufacture of medicated bandage adhesive strips.

The antibiofilm composition is believed, as a general proposition, but not necessarily to every listed species, to be active against:

(1) Gram Positive Bacteria, e.g., cacillus sp. (vegetative cell), Corynebacterium diptheriae, Micrococcus lutea, Micrococcus sp., Mycobacterium tuberculosis, Mycobacterium smegmatis, Propionibacterium acnes, Staphylococcus aureus, Staphylococcus epidermidis, Streptococcus faecalis, Streptococcus mutans, Streptococcus pneumonia, Streptococcus pyogenes;

(2) Gram Negative Bacteria, e.g., Acinetobacter calcoaceticus, Aeromonas hydrophilia, Citrobacter deversus, Citrobacter freundi, Enterobacter aerogenes, Enterobacter aglomerans, Enterobacter cloacae, Enterococcus, Escherichia coli, Klebsiella oxytoca, Klebsiella pneumoniae, Klebsiella terriena, Legionella pneumophila, Morganella morganii, Proteus mirabilis, Proteus vulgaris, Pseudomonas aeruginosa, Pseudomonas fluorscens, Salmonella cholera suis, Salmonella typhi, Salmonella typhimurium, Serratia liquifaciens, Serratia marcescens, Xanthomonas campestris;

In the field of use as a prophylactic against biofilm formation on an architectural inanimate surface (e.g., a floor and a shelf) and medical inanimate surfaces (e.g., implants and prostatics,) embodiments of the invention are used in the following manner. The antibiofilm composition of the present invention is generally capable of forming coatings on almost all inanimate surfaces. Preferably, the nature of the inanimate surface is known or ascertained and the inanimate surface is within one of the following categories of preferred inanimate surfaces. Preferred inanimate surfaces are siliceous materials, e.g., glass and sand; metals; textile fibers made from cotton, cellulose, acetate, polyester, nylon, wool, rayon and acrylon and plastics, e.g. nylon, polyvinyl chloride, fiberglass, epoxy, polyester and rayon.

Generally, the antibiofilm composition is used by applying it to an inanimate surface by misting, spraying, foaming, brushing or dipping (dipping is usually done with textiles.) The 3-(tri-methoxysilyl)propyldimethyl octadecyl ammonium chloride component of the composition, as discussed above, will react with oxides and hydroxyls on the surface of the inanimate surface through the above-described condensation reaction. The bonds formed are covalent and are resistant to re-hydration. On evaporation of the water solvent, a durable bonded coating is produced which last at least about 30 days.

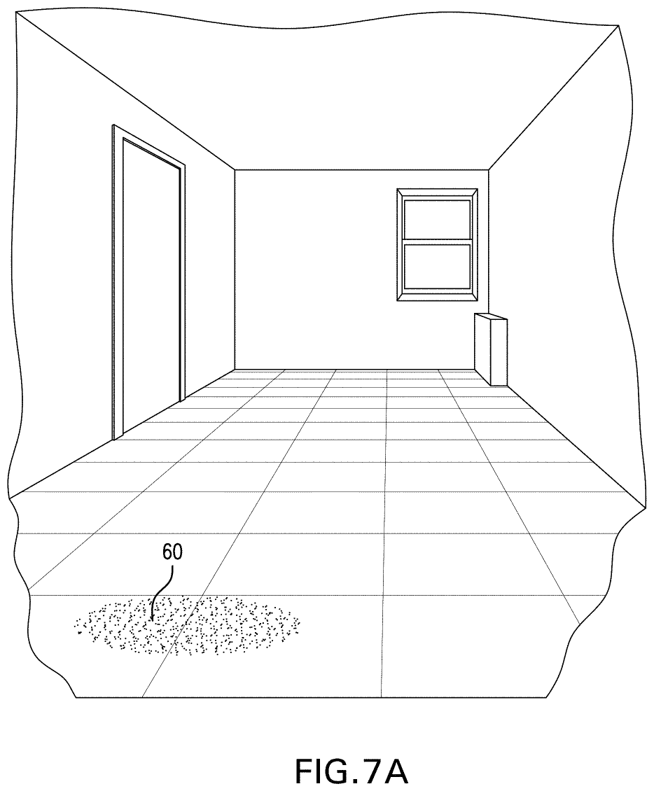

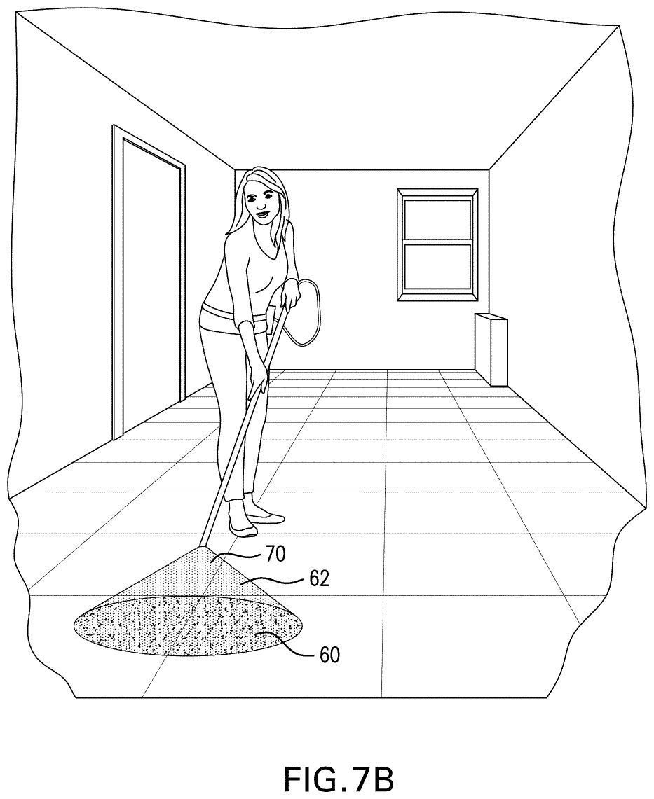

Referring to FIGS. 7A and 7B, in the field of use as a cleaning agent for an inanimate surface (e.g., a floor, a shelf) having on it and/or covered with the biofilm (60), embodiments of the invention are used in the following manner. A multiphase process is typically undertaken. The first phase is to apply an antibiofilm composition (70) according to the present invention to the inanimate surface. Methods for applying are spraying a liquid (62), spraying a foam (typically, an in situ generated foam where liquid composition in a storage vessel dispensed through an aspirating dispenser/pump nozel,) wiping, mopping and similar means, with liquid spraying and foam spraying preferred.

In a further phase, the benzalkonium component of the composition is allowed to act upon and penetrate the extracellular matrix resulting in the killing of bacteria in the biofilm. Likewise, the 3-(tri-methoxysilyl)propyldimethyl octadecyl ammonium chloride simultaneously acts upon and penetrates the matrix resulting in the killing of bacteria in the biofilm. In more detail, the benzalkonium chloride and the 3-(tri-methoxysilyl)propyldimethyl octadecyl ammonium chloride begin at the outermost surface of the biofilm to kill bacteria and break-up the biofilm. They proceed to penetrate the biofilm extracellular matrix and kill bacteria. This takes a period of about 1 minute to about two hours, depending upon how well-established are the bacteria in the biofilm (i.e., pervasiveness of the extracellular matrix,) the species of bacteria in the biofilm) the nature of the inanimate surface and temperature.

In a further phase, the 3-(tri-methoxysilyl)propyldimethyl octadecyl ammonium chloride is allowed to dry on the inanimate surface with the covalent bonding to form a positively charged micro thin coating on the inanimate surface which electrostatically clings negatively charged bacteria, pierces their outer membrane or coat and kills the microorganism, whereby for an extended period of between at least about 1 day to at least about 30 days bacteria are prevented from forming a biofilm.

Optionally and after the initial applying there is a further phase where the inanimate surface is cleaned by conventional means of soap and water (or water alone or other solvent) and wiping, sponging, mopping and the like to remove debris and otherwise put the inanimate surface in a cleaner state. Optionally, phases can be repeated; for example, applying, conventional washing and reapplying.

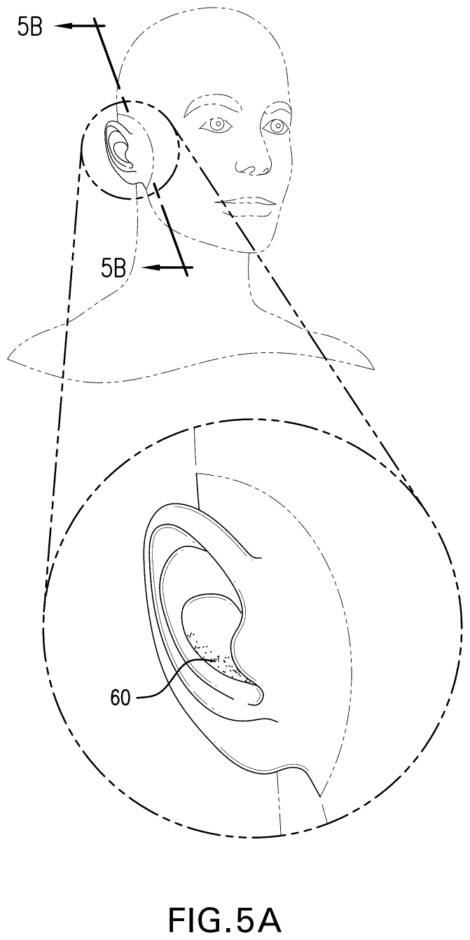

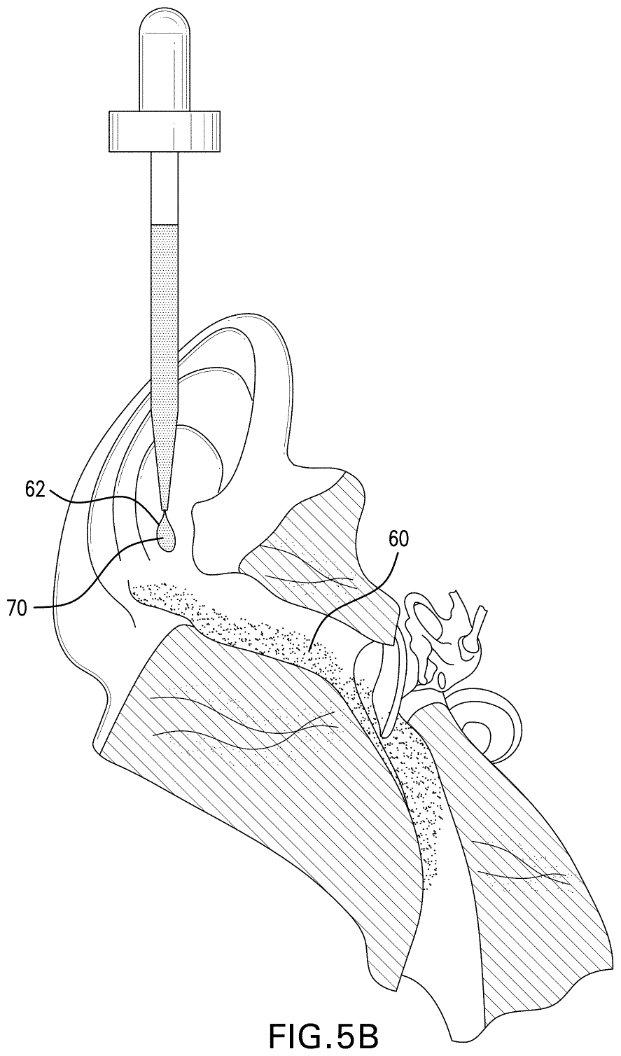

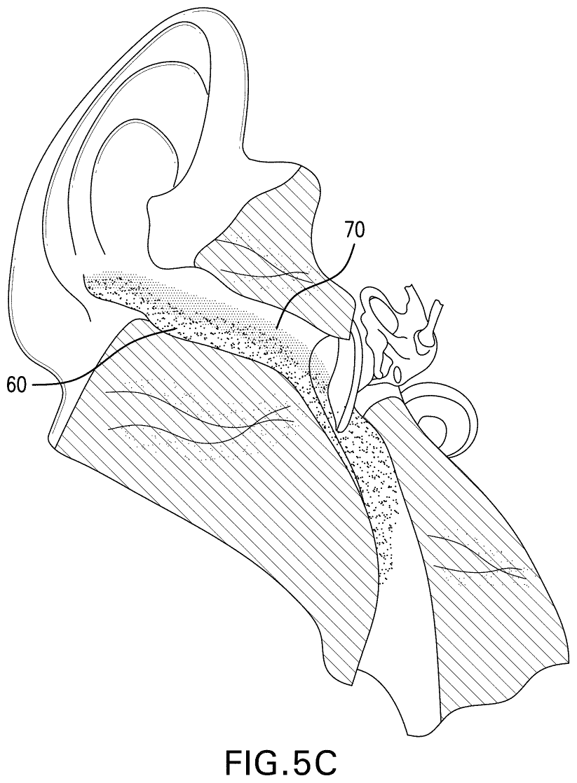

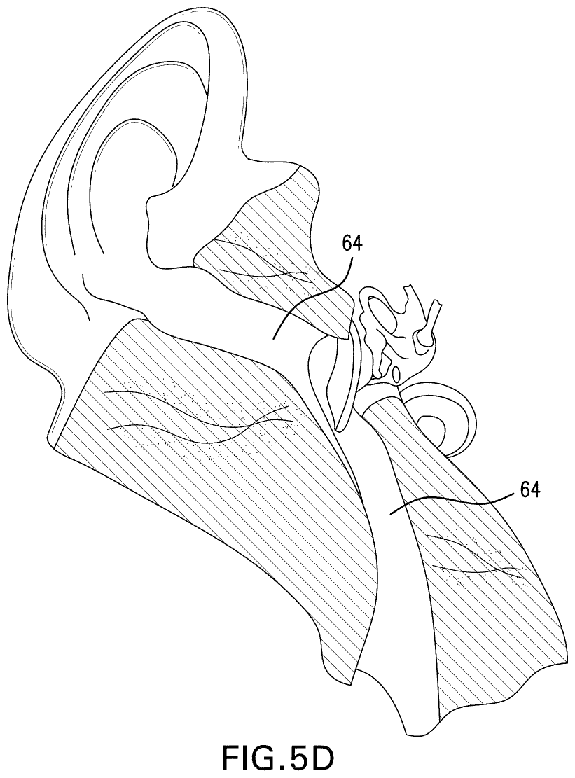

Referring to FIGS. 5A, 5B, 5C, 5D, 6A and 6B, at this point, there is a disclosure of use in the field of use as a medical treatment for an animate surface (namely, an epithelium and/or other tissue) that has biofilm (60) associated infirmity and in the subfield as a topical agent, embodiments of the invention are used in the following manner. Exemplary infirmities for treatment are a biofilm associated infirmity which is sinusitis, chronic rhinosinusitis, chronic suppurative otitis, post-sinus surgery infected sinuses, chronically infected middle ears associated with tympanic membrane perforation, a wound characterized by lacerated and/or deleteriously-altered skin and/or other tissue occasioned by trauma, a wound characterized by lacerated and/or deleteriously-altered skin and/or other tissue occasioned by a disease which does not allow soars to heal, a burn injury occasioned by heat, radiation, corrosive chemical and/or electricity which deleteriously altered skin and/or other tissue and acne disfiguration occasioned by oil build-up in blocked skin follicles.

A multi-phase process is typically undertaken. The first phase, is accommodating for treatment a living subject having an epithelium and/or other tissue that has biofilm (60) associated infirmity. Living subjects are human beings, horses, farm animals, zoo animals, dogs, cats, pets, mammals and other creatures having epithelium and/or other tissue that is infected by a biofilm. A preferred living subject is a human being.

The accommodating is accomplished by a positioning of the living subject for treatment. Typically, the living subject stations in a sitting posture on a chair, lying posture on a gurney or standing posture, with or without restraint. The accommodating can include receiving a living subject by a care giver (e.g., nurse, doctor) and the subject being in proximity to the care giver. A human living subject can self-accommodate himself.

In a further phase, there is ascertaining of the location of the epithelium and/or other tissue that has the biofilm (60) associated infirmity and that said location is assessable. Based on the above-described mode of action the antibiofilm compositions (70) of the present invention, there is no need to characterize the biofilm as to species of bacteria in the biofilm.

In a further phase, there is applying (62) to the located epithelium and/or other tissue an antibiofilm composition (70) according the present invention. Methods for applying and/or administering the antibiofilm composition (70) to an animate surface are localized bathing of a body part, generalized bathing of an entire body (not preferred,) intubation, irrigation and/or flushing with the antibiofilm composition (70) being an intubating/irrigating/cleansing solution, dripping (62), swabbing (e.g., using a cotton tip swab) and instillation from a bandage pad (68) function, or having a component functioning, as a reservoir which is impregnated with the antibiofilm composition (70). Preferred methods for applying are intubation, irrigation, dripping, swabbing and instillation from a bandage pad.

The application and/or administration is on a daily basis, multiple times per day basis or less than daily basis, depending upon desired and/or empirically achieved results. The duration of treatment is hours, days, weeks, months and/or ongoing, depending upon desired and/or empirically achieved results.

As for the particulars of a treatment regimen, consultation is to be made to the below examples and relevant portions of published clinical practice guides covering a particular condition (or multiple conditions) that is (are) relevant. The below clinical practice guides are incorporated herein by reference for the limited purposes of enablement with the proviso that as disclosed above, the antibiofilm composition functions in a manner that is agnostic to the particular microbes contained in the biofilm, such that characterization of the biofilm is an unneeded exercise. So incorporated by reference is (i) N. Hoiby, ESCMID guideline for the diagnosis and treatment of biofilm infections 2014, Elsevier Clinical Microbiology and Infection, Volume 21, Supplement 1, 1 May 2015, Pages S1-S25, doi: 10.1016/j.cmi.2014.10.024, Epub 2015 Jan. 14, https://www.sciencedirect.com/science/article/pii/S1198743X14000 901 and (ii) International Wound Infection Institute (IWII) Wound infection in clinical practice, Wounds International 2016, https://www.woundsme.com/uploads/resources/9b549b9d8a76B2c69a777 5Aa13157376.pdf.

While characterization of the antibiofilm composition is not needed, optionally, the biofilm can be characterized for the purpose of drawing an inference as to how much time will be taken for a biofilm infection to subside. Even though the compositions that are the subject of this invention are very, very broad spectrum, the kill rate varies with the species of bacteria. By characterizing the bacteria in a biofilm infection, a deduction is made as to the treatment duration for an infection to subside.

The previously described versions of the present invention have many advantages. One advantage is an ability to effectively penetrate the extra-cellular polymer matrix of biofilms in both the settings of inanimate surfaces (e.g., shelves and floors) and animate surfaces (epithelium and other tissue.)

Another advantage is penetrating a biofilm in a manner so as after penetrating being potent to kill bacteria.

A related advantage in the medical field is a kill effectiveness at a level that controls or eradicates a biofilm.

Another advantage is being extremely well tolerated on human mucosal surfaces.

Another advantage is killing by a physicochemical mode of action of rupturing bacterial outer membranes or cell walls.

Related advantages are being broad spectrum to nearly universal and not promoting the evolution of super resistant bacteria.

A related advantage is surpassing the ability of bacteria to mutate into resistant strains so as not to contribute to the evolution of super microbes that cannot be killed.

Another related advantage is the treatment biofilm infected animate surface (e.g., epithelium and other tissue) without necessarily having to characterize the bacteria in the biofilm. While the compositions according to this invention are very, very broad spectrum, the kill rate varies with species of bacteria. Optionally, the biofilm can be characterized to ascertain the species of infecting bacteria so as to make a prediction of the time for an infection to subside.

Another advantage is a long mode of action preventing additional replication of microbes remaining after the initial kill.

Another advantage is a longer shelf life with respect to prior art compositions, including a shelf life of at least about three-years boding to about four-years. This enhances the commercial viability of a product.

### EXAMPLES

The following examples further describe and demonstrate embodiments within the scope of the present invention. The examples are given solely for the purpose of illustration and are not to be construed as limitations or restrictions of the present invention, as persons skilled in the art will quickly realize many variations thereof are possible that are all within the spirit and scope of the invention.

### Example 1

### General Bacterial Biocidal Activity

Example 1 is testing having the objective of determining as a general matter (i.e, not bacteria in a biofilm) the antibacterial efficacy of an antibiofilm composition comprised of about 0.10% (w/w) benzalkonium chloride; about 0.50% (w/w) phenoxyethanol; about 0.20% (w/w) 3-(tri-methoxysilyl)propyldimethyl octadecyl ammonium chloride and about 99.2% (w/w) deionized water. The testing methodology is in accordance with BSEN 1276. The test involved the preparation of a standard suspension of test organisms containing 1.5-5.0×108 cells per ml (milliliter) of the following four standard bacteria: Escherichia coli K12 NCTC 10538, Enterococcus hirae ATCC 10541, Pseudomonas aeruginosa ATCC 15442, Staphylococcus aureus ATCC 6538 and Salmonella typhimurium ATCC 13311.

In overview, the test procedure was as follows. A sample of antibiofilm composition was delivered or diluted to a test suspension of bacteria in a solution of an interfering substance. The mixture was maintained at 20 degrees Celsius for 24 hrs. At the end of this contact time an aliquot was taken and the bactericidal or bacteriostatic activity in this portion was immediately neutralized or suppressed by a validation method. After 5 mins (minutes) of neutralization time 1 mL (milliliter) of the sample mixture was taken and there was performance of the pour plate method. Further, there was a keeping for incubation at 37° C. (degrees Celsius) for 48 hrs (hours). A dilution neutralization was the method of choice. The numbers of the surviving bacteria in the sample were determined and the reduction was calculated.

Particular experiment conditions are presented in the below table:

| | |
|---|---|
| Clean condition | 0.3 g/L (grams per liter): |
| | Bovine Serum Albumin |
| Agar | Tryptone Soya Agar |
| Neutralizers | Polysorbate 80, 30 g/l |
| | (grams per liter) + |
| | lecithin, 3 g/l + Sodium |
| | thioglycolate 30 g/L prepared |
| | in de-ionized water and |
| | autoclaved for 15 minutes at |
| | 121° C. (degrees Celsius) |

On the basis of the calculated reduction results it can be concluded that the tested specimen was observed with a 6 log reduction in bacterial count at 24 hrs (hours) contact time against Escherichia coli K12, Enterococcus hirae, Pseudomonas aeruginosa, Staphylococcus aureus, and Salmonella typhimurium.

### Example 2

### General Bacterial Biocidal Activity

Example 2 is an evaluation of the general activity (not bacteria in a biofilm) according to PN-EN 13727+A2 of an antibiofilm composition comprised of about 0.10% (w/w) benzalkonium chloride; about 0.50% (w/w) phenoxyethanol; about 0.20% (w/w) 3-(tri-methoxysilyl)propyldimethyl octadecyl ammonium chloride and about 99.2% (w/w) deionized water. The test method was dilution-neutralization. The antibiofilm composition diluent in the test was distilled water. Concentrations tested were 1%-97% v/v (percent by volume.) The interfering substance was 0.3 g/l (grams per liter) bovine albumin. The test temperature was 20.0° C.+/−1.0° C. (degrees Celsius.) The contact time was 60 s+/−5 s (seconds.) The incubation parameters were 37.0° C.+/−1.0° C. (degrees Celsius,) 48 h (hours) and pour plate method. The microbial strains in the test were Pseudomonas aeruginosa ATCC 15442, Staphylococcus aureus ATCC 6538, Enterococcus hirae ATCC 10541 and Escherichia coli K12 NCTC 10538.

The conclusion from the test results is that the antibiofilm composition according to PN-EN 13727+A2:2015-12 at clean conditions (0.3 g/l bovine albumin), test temperature 20.0° C.±1.0° C., contact time 60 s, diluted in distilled water, is active (necessary reduction 5 log) against Pseudomonas aeruginosa ATCC 15442 at 97% v/v, Staphylococcus aureus ATCC 6538 at 97% v/v, Enterococcus hirae ATCC 10541 at 97% v/v and Escherichia coli K12 NCTC 10538 at 97% v/v.

### Example 3

### Mature Biofilm Growth on Coupons

In this Example 3, mature biofilms were established on polycarbonate coupons (32) using a CDC biofilm reactor (30). Determinations in Example 4 herein used these mature established biofilms on polycarbonate coupons.

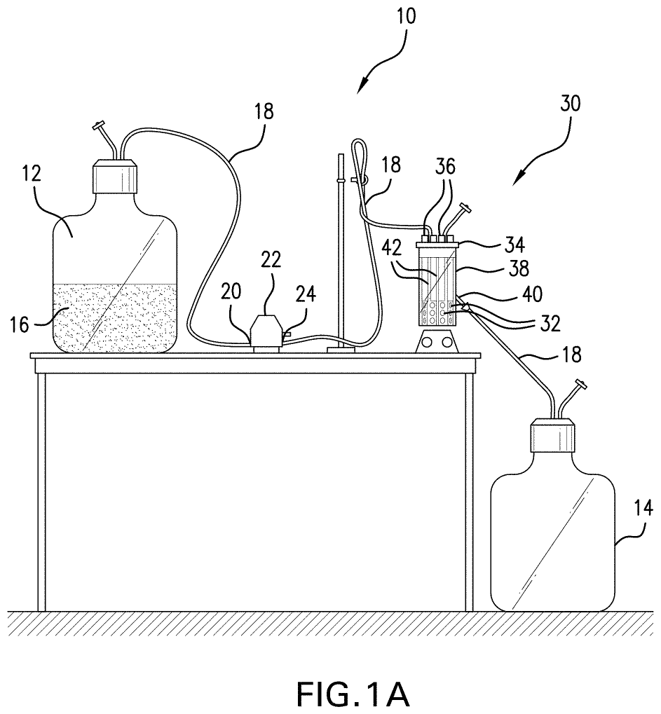

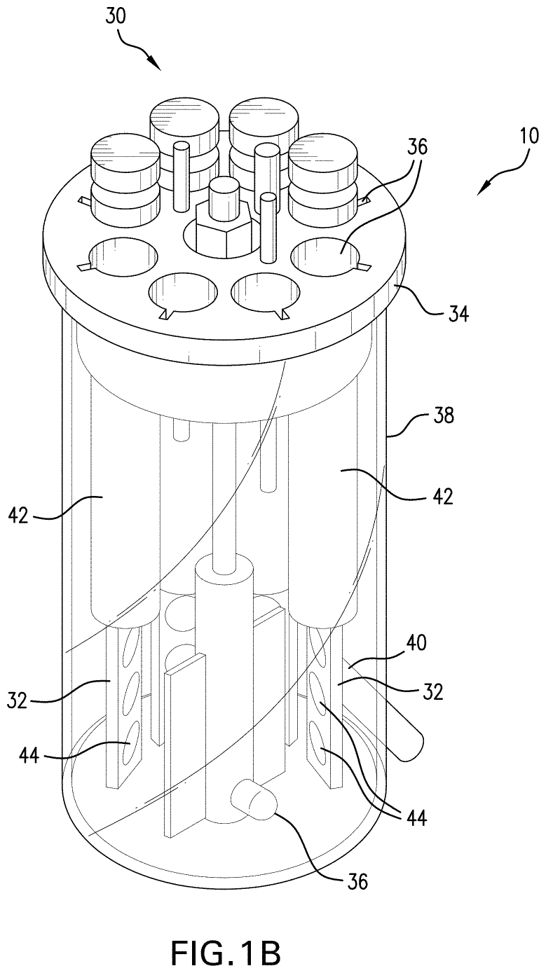

Referring to FIGS. 1A and 1B, there is illustrated a CDC Biofilm Reactor (30) by Biosurface Technologies Corporation (Bozeman, Mont., USA) (CDC-BR) situated in an operational setup (10). Referring to FIG. 1A, the operational setup (10) for a CDC-BR (30) is comprised of a first tank (12) (also called a medium tank) holding circulation medium (16); said first tank (12) is in fluid communication via tubing (18) with the inlet (20) of a pump (22) having an outlet (24); the outlet is in fluid communication via tubing (18) with ports (36) in a ported lid (34) of a reactor vessel (38) having a lower outlet (40) and the lower outlet (40) is in fluid communication via tubing (18) with a second tank (14) (also called a waste tank) for holding waste.

Referring to FIG. 1B, the reactor vessel (38) in pertinent part is associated with and/or comprised of a ported lid (34), a vessel (38), coupon holders (42), removable growth substrate (44) circular component of the coupon (32) (about 12.7 mm (mm meaning millimeter,) an outlet for waste (40) and a magnetic stir bar (46). There are also seals, a coupon removal tool, a coupon manipulation tool, and glass flow breaks, each of which it is not illustrated.

Bacterial strains were Obtained. In particular, the below bacterial strains were obtained:

(i) ATCC (American Type Culture Collection) strain 6538 Staphylococcus aureus (S. aureus) subsp. Aureus Rosenbach;

(ii) ATCC strain 27853 (Pseudomonas aeruginosa (P. aeruginosa) and

(iii) ATCC 27586 Pseudomonas aeruginosa (Schroeter) Migula. Each of the bacterial strain were known biofilm formers and comply with international standards (discussed further below.) The growth of a clinical Staphylococcus aureus isolate from a chronic sinusitis patient on polycarbonate coupons in tryptic soy broth was previously established. Accordingly, this strain was selected for use in certain of the below determinations.

The method for establishing mature biofilms was based on the “Standard Test Method for Quantification of Pseudomonas aeruginosa Biofilm Grown with High Shear and Continuous Flow using CDC Biofilm Reactor” (ASTM E2562) and the standard method for “Growing a Biofilm using the CDC Biofilm Reactor” (USEPA MB-19). One milliliter (mL) of fresh S. aureus or P. aeruginosa overnight culture (109 CFU/mL) was inoculated into 350 mL of tryptic soy broth (TSB) in the reactor (30). The biofilm reactor (30) was placed on a magnetic hotplate stirrer (not illustrated) and incubated in batch mode at 34° C. (C being Celsius) and a rotational speed of 120 rpm (rpm being rotations per minute) for 24 h (h being hour) to encourage cell adhesion.

At this time point, a continuous flow of 20% TSB through the reactor was initiated and maintained at 11.1 mL/min (mL/min the milliliters per minute) for an additional 24 h to allow for biofilm expansion. Continuous flow was sustained for an extra 24 h with 10% TSB to stimulate maturation of the biofilm. After a total 72 h of biofilm growth, 5 L (L being liter) of saline were flushed through the reactor at approximately 25 mL/min to wash out planktonic cells from the system.

### Example 4

### Testing Against Biofilms

Example 4 is an evaluation (testing) of disinfecting efficacy against biofilms of a tested composition. The tested composition was comprised of (a) about 0.10% (w/w) (w/w being percentage by weight) benzalkonium chloride; (b) about 0.20% (w/w) 3-(tri-methoxysilyl)propyldimethyl octadecyl ammonium chloride; (c) about 0.50% (w/w) phenoxyethanol and (d) about 99.2% (w/w) deionized water.

The method for testing was based on the “Standard Test Method for Evaluating Disinfectant Efficacy against Pseudomonas aeruginosa Biofilm Grown in CDC Biofilm Reactor using Single Tube Method” (ASTM E2871) and the “Single Tube Method for Determining the Efficacy of Disinfectants against Bacterial Biofilm” (USEPA MB-20). From an overall perspective, the determination protocols included time point 0 (baseline) controls, individual treatment controls for each antimicrobial contact time, a positive control of the bacterial inoculum as well as negative controls for the reactor and plating media and the above described tested antibiofilm composition.

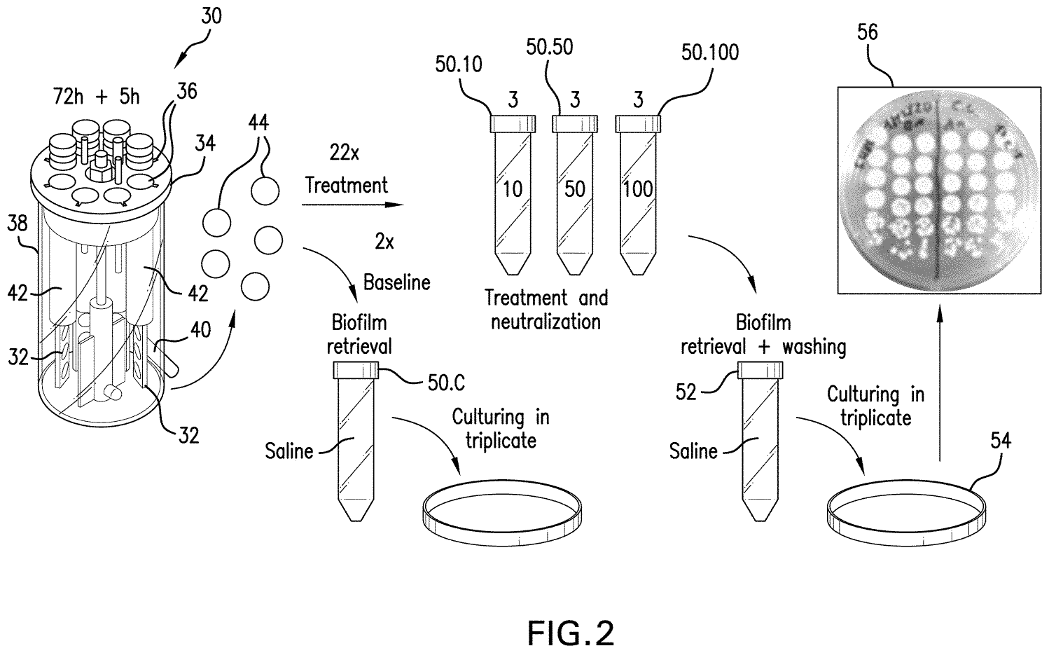

Referring to FIG. 2, sample coupons (44) prepared in Example 3 were removed from the CDC biofilm reactor (30) under aseptic conditions. Composition concentrations of 10%, 50% and 100% were tested. Since the above-described anti-biofilm composition being tested is a product containing the active ingredients rather than the active ingredient in its natural form, this necessitated working with the percent of the product with 100% being the maximum concentration that can be tested, treatment medium contained normal saline with 10%, 50% or 100% of the above described antibiofilm composition.

The sample coupons (44) were individually distributed into 50 mL (mL being milliliter) centrifuge tubes (Falcon tubes) (50.10, 50.50 and 50.100) containing 2 mL treatment solution. The determination protocol used 0.9% NaCl solution (normal saline) as the treatment medium. Two tubes with 2 mL saline served as control treatment for each time point in each run. In sum, 3 coupons per treatment group and 2 coupons each for baseline and treatment controls.

Generally, biofilms were incubated at room temperature for between 5 min and overnight (20-21 hours) according to the parameters specified in the matrixes contained in the below tables (or move on immediately for baseline.) During treatment all coupons (plates) are stationary

More specifically, set out in the below table is the S. aureus biofilm treatment matrix with number of coupons (replicates) per treatment and where the overnight treatment was between 20 and 21 hours.

| | 5 min | 1 h | 6 h | overnight |
|---|---|---|---|---|
| 10% | 8 | 9 | 9 | 9 |
| 50% | 8 | 9 | 9 | 8 |
| 100% | 9 | 8 | 9 | 9 |

More specifically, set out in the below table is the P. aeruginosa biofilm treatment matrix with number of coupons per treatment.

| | 5 min | 1 h | 6 h | overnight |
|---|---|---|---|---|
| 10% | 9 | 9 | 9 | 9 |
| 50% | 9 | 9 | 9 | 9 |
| 100% | 9 | 9 | 9 | 9 |

Continuing to refer to FIG. 2, after the set contact time, the activity of the composition or deluded composition was inhibited immediately by transferring each coupon into a fresh tube (52) with 3 mL D/E medium (meaning Dey/Engley neutralisation medium (also called a solution or broth) and incubating for at least 1 min. Continuing to refer to FIG. 2, subsequently, coupons were briefly dipped (or suspended) into saline to remove (wash off) any residual D/E medium before transferring them into fresh tubes (52) containing 5 mL saline.

Biofilms were then retrieved (harvested) from coupons by shaking at a speed of 2.4 m/s (m/s meaning meters per second) for 1 min, sonication for 10 min (80 Hz with Hz meaning Hertz) and shaking for an additional 1 min. The remaining bacterial solution was 10-fold serial diluted and plated in triplicate on tryptic soy agar (TSA) (54) (namely, dried tryptic soy agar) using the spot-plating technique (namely, plating 10 ul.) Series dilution and plating was carried out by (a) preparation a 96-well flat bottom culture plate with 90 uL normal saline in rows 2-12 for dilution series; (b) a transfer 3×100 ul (microliter) biofilm solution from each sample into the first row of the 96-well plate and (c) performance of 10× dilutions by transferring 10 uL from the first row of wells to the next and so on so forth.

Continuing to refer to FIG. 2, TSA plates were incubated at 37° C. overnight and the total amount of biofilm recovered from each coupon was determined by colony counts (CFU/coupon) (namely, determining total number of recovered colony forming units CFUs.) Data was gathered from plates (56) having 12 independent biofilm runs, each yielding 24 coupons (replicates) that were split into treatment groups. Two coupons in each run underwent biofilm retrieval and bacterial enumeration immediately after being removed from the reactor to determine the baseline amount of biofilm per coupon (baseline control.)

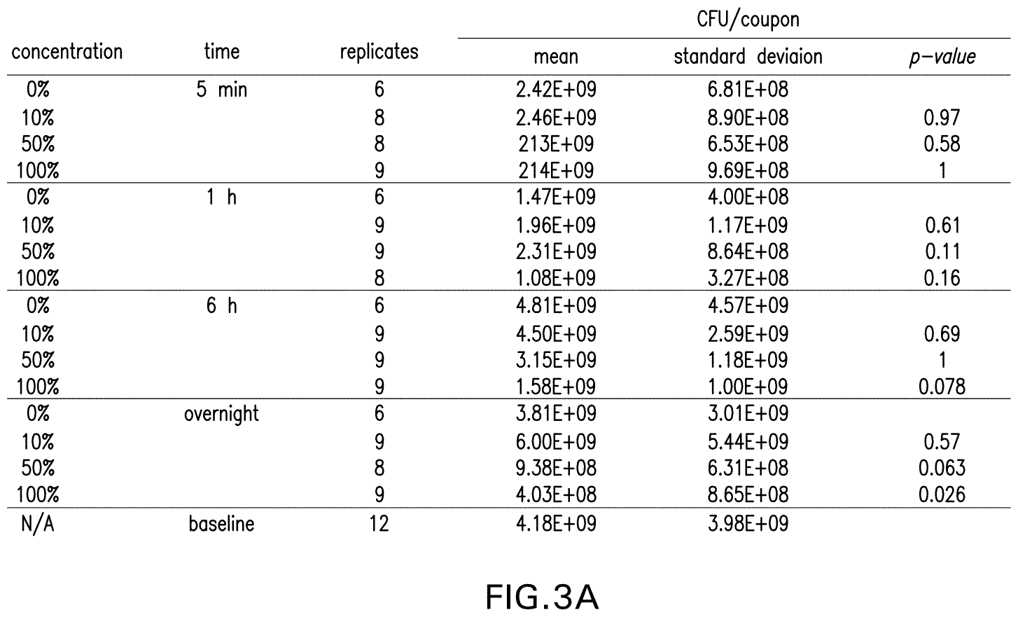

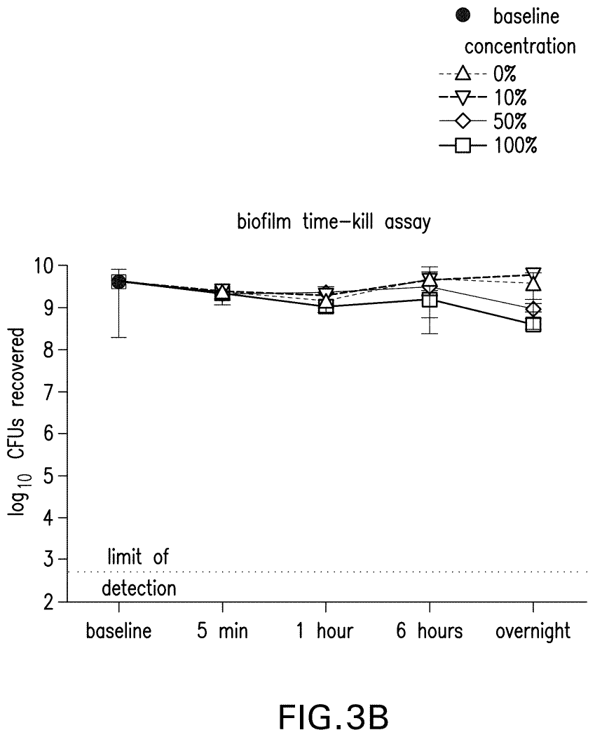

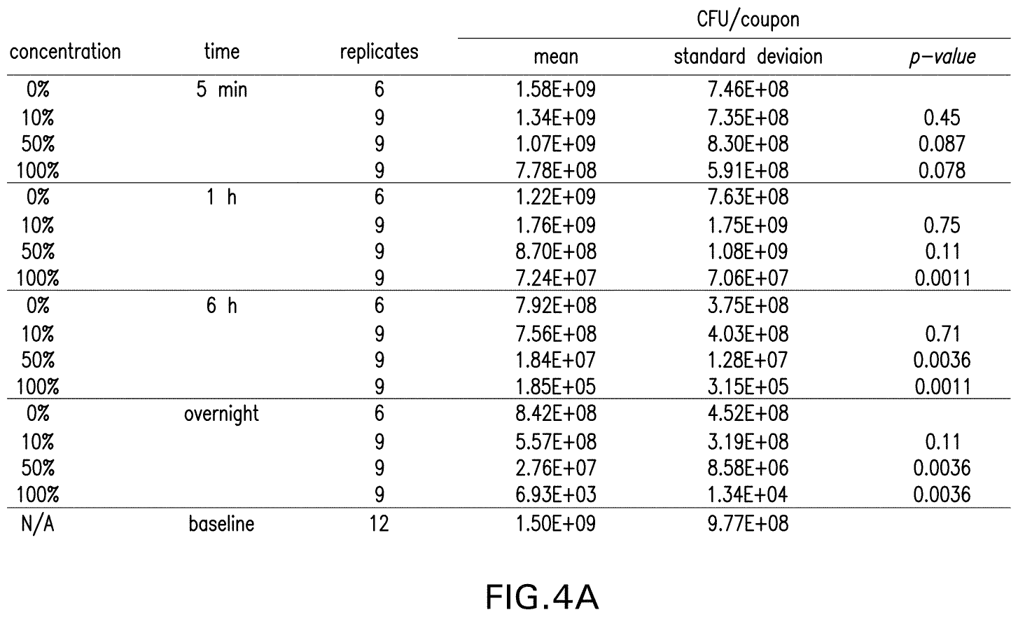

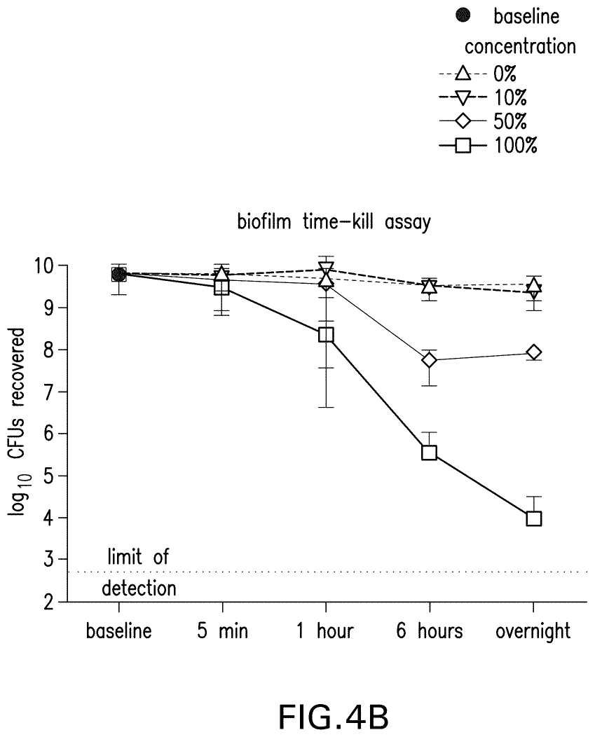

Referring to FIGS. 3A and 4A, colony counts were summarised as means+/−standard deviation. Coupons for which colony counts could not be determined due to experimental complications were removed from the dataset. The Pairwise-Wilcoxon test with Benjamini-Hochberg correction for multiple comparisons was performed to determine significant reductions in biofilm between control (saline) and composition/diluted composition treatments.

Continuing to refer to FIG. 3A and also referring to FIG. 3B, the overall findings indicate significant antibiofilm activity of the tested composition against P. aeruginosa. Against S. aureus, overnight treatment with 100% composition resulted in an approximate 1 log₁₀ reduction in viable cells. FIG. 3A is a tabular showing of data on colony forming units (CFUs) recovered after treating S. aureus biofilms. P-values <0.05 indicate a significant difference from controls. FIG. 3B is a graphical showing summarizing the results of the time-kill experiments on S. aureus ATCC 6538 biofilms. It shows the log₁₀ of total CFUs recovered from each coupon after different contact times of various concentrations of the tested composition. As per the key table, each icon of a particular geometric shape represents a different treatment concentration. Baseline controls from all runs are summarized as a single data point (circle). The limit of detection (dotted line) represents the lowest number of cells per coupon that could be detected using this assay.

Referring to FIGS. 4A and 4B, against P. aeruginosa, treatment at a 50% concentration of the tested composition resulted in an approximate 2 log₁₀ reduction at 6 h and overnight. Treatment with 100% tested composition resulted in an approximate 1 log₁₀ reduction at 1 h, ˜4 log₁₀ (˜ means approximate) reduction at 6 h, and ˜5 log₁₀ reduction (i.e. 99.999% killing) with overnight treatment. FIG. 4A is a tabular showing of data on CFUs recovered after treating P. aeruginosa biofilms at certain concentrations of the tested composition. P-values <0.05 indicate a significant difference from controls.

Referring to FIG. 4B, there is a graphical showing summarizing the results of the time-kill experiments on P. aeruginosa ATCC 27586 biofilms. It shows the log₁₀ of total CFUs recovered from each coupon after different contact times of various concentrations of the tested composition. As per the key table, each icon of a particular geometric shape represents a different treatment concentration. Baseline controls from all runs are summarized as a single data point (circle). The limit of detection (dotted line) represents the lowest number of cells per coupon that could be detected using this assay.

The tested composition is highly effective against biofilms of the Gram-negative organism P. aeruginosa in vitro. Results from testing the tested composition against biofilms of the Gram-positive organism S. aureus in vitro suggest a trend towards increasing antibiofilm efficacy with durations of exposure greater than those tested here.

### Example 5

### Treatment for Post-Sinus Surgery Biofilm Infected Sinuses

Example 5 is hypothetical treatment regimen for post-sinus surgery biofilm infected sinuses. This condition could be treated by twice or three times daily application of a foam or spray of the antibiofilm composition comprised of between about 0.05% (w/w) to about 0.20% (w/w) benzalkonium chloride; between about 0.20% (w/w) to about 0.50% (w/w) 3-(tri-methoxysilyl)propyldimethyl octadecyl ammonium chloride; between about 0.25% (w/w) to about 0.75% (w/w) phenoxyethanol and between about 98.55% (w/w) to about 99.5% (w/w) deionized water. Treatment is continued until the infection subsides.

### Example 6

### Treatment Regimen for a Chronically Infected Middle Ears

Referring to FIGS. 5A-5D, Example 6 is a hypothetical treatment regimen for chronically infected middle ears (60) with tympanic membrane perforation. This condition could be treated by twice or three times daily application of foam, spray or droplets (62) of an antibiofilm composition comprised of between about 0.05% (w/w) to about 0.20% (w/w) benzalkonium chloride; between about 0.20% (w/w) to about 0.50% (w/w) 3-(tri-methoxysilyl)propyldimethyl octadecyl ammonium chloride; between about 0.25% (w/w) to about 0.75% (w/w) phenoxyethanol and between about 98.55% (w/w) to about 99.5% (w/w) deionized water. The treatment is continued until the infection subsides (64).

### Example 7

### Treatment of a Biofilm Infected Arm Wound with a Bandage

Example 7 is hypothetical treatment of biofilm infected arm. Referring to FIG. 6A, a human subject is afflicted with an arm wound which became infected and were said infection progressed to being a biofilm (60). Due to the infecting microbes having reached the stage of forming a biofilm, the infection has become chronic with any healing arrested. Without having first taken a biopsy or otherwise characterized the microbe construct of the biofilm, a nurse proceeds to treat the wound by dressing and closing it with a bandage (66) according the present invention.

Referring to FIG. 6B, the bandage (66) being put on the human subject's arm has a pad (68) which is impregnated with an antibiofilm composition (70) comprised of between about 0.05% (w/w) to about 0.20% (w/w) benzalkonium chloride; between about 0.20% (w/w) to about 0.50% (w/w) 3-(tri-methoxysilyl)propyldimethyl octadecyl ammonium chloride; between about 0.25% (w/w) to about 0.75% (w/w) phenoxyethanol and between about 98.55% (w/w) to about 99.5% (w/w) deionized water. Referring to FIG. 6C, as with any medicated dressing, by way of osmotic, hydrodynamic and physical forces, the antibiofilm composition impregnated in the pad (68, 70) is applied to the wound.

The bandage (66) is periodically changed on a daily basis until the biofilm infection (60) subsides.

### Example 8

### Cleaning a Floor Ladened with a Biofilm

Example 8 is a hypothetical example of cleaning a floor laden with a biofilm. Referring to FIG. 7A, a floor has on it a biofilm (60). Due to the biofilm, the floor is slimy/slippery (or sticky) discolored/stained. The biofilm is spreading. Referring to FIG. 7B, spray applied to the floor is an antibiofilm composition (62, 70) of between about 0.05% (w/w) to about 0.20% (w/w) benzalkonium chloride; between about 0.20% (w/w) to about 0.50% (w/w) 3-(tri-methoxysilyl)propyldimethyl octadecyl ammonium chloride; between about 0.25% (w/w) to about 0.75% (w/w) phenoxyethanol and between about 98.55% (w/w) to about 99.5% (w/w) deionized water. The antibiofilm composition (70) begins at the surface of the biofilm to break up the biofilm and progresses to penetrate the film. The next day, the floor is conventionally mopped clean. The antibiofilm composition (70) is reapplied (62) as a prophylactic measure. Thirty days later the floor in next conventionally cleaned by mopping with soapy water and thereafter, the antibiofilm composition reapplied.

### Example 9

### Prophetic Aging Study

Example 9 is a prophetic aging study. Hypothetically, there is an acquisition of a plurality of bottles and/or packages of composition according to the present invention. Each bottle and/or package is in the manner of what is being customarily sold and/or distributed. About half of this plurality of bottles and packages are stored in a warehouse and subjected to the standard operating conditions of the warehouse; that is, variations in temperature, humidity, lighting etc. The other about half of the plurality of bottles and packages are stored in retail stores, hospital stock rooms and cleaning supply cabinets and closets. Likewise, this plurality of bottles and packages are subjected to the standard operating conditions of the places where they are being stored; i.e., variations in temperature, humidity, lighting etc.

After 36 months and 48 months elapse, the compositions according to the present invention in the stored bottles and packages are evaluated (tested) in the manner of examples 3 and 4. Forecasted is that the results will show that the compositions are highly effective against P. aeruginosa biofilms with an approximate 5 log₁₀ (99.999%) reduction in viable bacteria after overnight treatment and effective against S. aureus with an approximate 1 log₁₀ reduction being observed after overnight treatment. In the alternative, a modestly lower, but not significantly lower, effectiveness from the foregoing.

Although the present invention has been described in considerable detail with reference to certain preferred versions thereof, other versions are possible with substituted, varied and/or modified materials and steps are employed. These other versions do not depart from the invention. Therefore, the spirit and scope of the appended claims should not be limited to the description of the preferred versions contained herein.

What is claimed is:

1. An antibiofilm composition comprised of:
a between about 0.05% (w/w) to about 0.20% (w/w) benzalkonium chloride;
b between about 0.20% (w/w) to about 0.50% (w/w) 3-(tri-methoxysilyl)propyldimethyl octadecyl ammonium chloride;
c between about 0.25% (w/w) to about 0.75% (w/w) phenoxyethanol and
d between about 98.55% (w/w) to about 99.5% (w/w) deionized water.

2. The antibiofilm composition of claim 1 where the:
a the benzalkonium chloride is that of the same which meets the description of Chemical Abstracts Service No. 68424-85-1;
b the 3-(tri-methoxysilyl)propyldimethyl octadecyl ammonium chloride is that of the same which meets the description of Chemical Abstracts Service No. 27668-52-6
c the phenoxyethanol is that of the same which meets the description of Chemical Abstracts Service No. 122-99-6 and
d the deionized water is that of the same which meets the description of Chemical Abstracts Service No. 7732-18-5.

3. The antibiofilm composition of claim 1 having between about 0.05% (w/w) to about 0.15% (w/w) fragrance with there being proportionally less deionized water such that the weight percentage total of all the ingredients is 100%.

4. The antibiofilm composition of claim 3 where the fragrance is selected from the group consisting of bergamot, gardenia, honeysuckle, jasmine, lavender, pear, peony, rosemary, sandalwood and vanilla.

5. A wound dressing comprised of:
(i) a bandage component and
(ii) a pad component that has as a reservoir capability which is impregnated with the antibiofilm composition of claim 1,
whereby the antibiofilm composition in the reservoir is communicable to epithelium and/or other tissue having a biofilm associated infirmity.

6. A method for cleaning an inanimate surface having on it a biofilm with prophylaxis comprised of the steps of:
applying to the inanimate surface the antibiofilm composition of claim 1;
a allowing the benzalkonium chloride to penetrate the biofilm extracellular matrix and kill bacteria;
b allowing the 3-(tri-methoxysilyl)propyldimethyl octadecyl ammonium chloride to penetrate the biofilm extracellular matrix and kill bacteria and dry on the inanimate surface with the covalent bonding of the 3-(tri-methoxysilyl)propyldimethyl octadecyl ammonium chloride to form a micro thin coating on the surface,
whereby for an extended period of between at least about 1 day to at least about 30 days bacteria are effectively prevented from existing in a biofilm.

7. The method of claim 6 where the applying is by liquid spraying or foam spraying.

8. A prophylactic method against the formation of a biofilm on an inanimate surface comprised of the steps of:
applying to the inanimate surface the antibiofilm composition of claim 1;
a allowing the 3-(tri-methoxysilyl)propyldimethyl octadecyl ammonium chloride to dry on the inanimate surface with the covalent bonding of the 3-(tri-methoxysilyl)propyldimethyl octadecyl ammonium chloride to form a micro thin coating on the surface,
whereby for an extended period of between at least about 1 day to at least about 30 days bacteria are effectively prevented from existing in a biofilm.

9. A method of treating a living subject having an epithelium and/or other tissue that is infected by a biofilm associated infirmity comprised of the steps of:
a accommodating for treatment a living subject having epithelium and/or other tissue that has biofilm associated infirmity;
b ascertaining the location of the epithelium and/or other tissue that has the biofilm associated infirmity and that said location is assessable and
applying to the located epithelium and/or other tissue the antibiofilm composition of claim 1 until the infection subsides.

10. The method of claim 9 where the living subject is a human being.

11. The method of claim 9 where the applying is done by a process selected from the group consisting of dripping, foaming, instilling from a bandage pad, irrigating, swabbing and spraying.

12. The method of claim 9 were the biofilm associated infirmity being treated is sinusitis, chronic rhinosinusitis, chronic suppurative otitis, post-sinus surgery infected sinuses, chronically infected middle ears associated with tympanic membrane perforation, a wound characterized by lacerated and/or deleteriously-altered skin and/or other tissue occasioned by trauma, a wound characterized by lacerated and/or deleteriously-altered skin and/or other tissue occasioned by a disease which does not allow soars to heal, a burn injury occasioned by heat, radiation, corrosive chemical and/or electricity which deleteriously altered skin and/or other tissue and acne disfiguration occasioned by oil build-up in blocked skin follicles.

13. The method of claim 9 where the applying is twice or three times daily by foam or spray until the infection subsides.

14. An antibiofilm composition comprised of:
a about 0.10% (w/w) benzalkonium chloride;
b about 0.20% (w/w) 3-(tri-methoxysilyl)propyldimethyl octadecyl ammonium chloride;
c about 0.50% (w/w) phenoxyethanol and
d about 99.2% (w/w) deionized water.

15. The antibiofilm composition of claim 14 where the:
a the benzalkonium chloride is that of the same which meets the description of Chemical Abstracts Service No. 68424-85-1;
b the 3-(tri-methoxysilyl)propyldimethyl octadecyl ammonium chloride is that of the same which meets the description of Chemical Abstracts Service No. 27668-52-6
c the phenoxyethanol is that of the same which meets the description of Chemical Abstracts Service No. 122-99-6 and
d the deionized water is that of the same which meets the description of Chemical Abstracts Service No. 7732-18-5.

16. The antibiofilm composition of claim 14 having about 0.1% (w/w) fragrance with there being about 99.1 (w/w) deionized water such that the weight percentage total of all the ingredients is 100%.

17. The antibiofilm composition of claim 14 where the fragrance is selected from the group consisting of bergamot, gardenia, honeysuckle, jasmine, lavender, pear, peony, rosemary, sandalwood and vanilla.

18. A wound dressing comprised of:
(i) a bandage component and
(ii) a pad component that has as a reservoir capability which is impregnated with the antibiofilm composition of claim 14,
whereby the antibiofilm composition in the reservoir is communicable to epithelium and/or other tissue having a biofilm associated infirmity.

19. A method for cleaning an inanimate surface having on it a biofilm with prophylaxis comprised of the steps of:
a applying to the inanimate surface the antibiofilm composition of claim 14;
b allowing the benzalkonium chloride to penetrate the biofilm extracellular matrix and kill bacteria;
c allowing the 3-(tri-methoxysilyl)propyldimethyl octadecyl ammonium chloride to penetrate the biofilm extracellular matrix and kill bacteria and dry on the inanimate surface with the covalent bonding of the 3-(tri-methoxysilyl)propyldimethyl octadecyl ammonium chloride to form a positively charged micro thin coating on the surface,
whereby for an extended period of between at least about 1 day to at least about 30 days bacteria are effectively prevented from existing in a biofilm.

20. The method of claim 19 where the applying is by liquid spraying or foam spraying.

21. A prophylactic method against the formation of a biofilm on an inanimate surface comprised of the steps of:
a applying to the inanimate surface the antibiofilm composition of claim 14;
b allowing the 3-(tri-methoxysilyl)propyldimethyl octadecyl ammonium chloride to dry on the inanimate surface to form a micro thin coating on the surface,
whereby for an extended period of between at least about 1 day to at least about 30 days bacteria are effectively prevented from existing in a biofilm.

22. A method of treating a living subject having an epithelium and/or other tissue that is infected by a biofilm associated infirmity comprised of the steps of:
a accommodating for treatment a living subject having epithelium and/or other tissue that has biofilm associated infirmity;
b ascertaining the location of the epithelium and/or other tissue that has the biofilm associated infirmity and that said location is assessable and
c applying to the located epithelium and/or other tissue the antibiofilm composition of claim 14 until the infection subsides.

23. The method of claim 22 where the living subject is a human being.

24. The method of claim 22 where the applying is done by a process selected from the group consisting of dripping, foaming, instilling from a bandage pad, irrigating, swabbing and spraying.

25. The method of claim 22 were the biofilm associated infirmity being treated is sinusitis, chronic rhinosinusitis, chronic suppurative otitis, post-sinus surgery infected sinuses, chronically infected middle ears associated with tympanic membrane perforation, a wound characterized by lacerated and/or deleteriously-altered skin and/or other tissue occasioned by trauma, a wound characterized by lacerated and/or deleteriously-altered skin and/or other tissue occasioned by a disease which does not allow soars to heal, a burn injury occasioned by heat, radiation, corrosive chemical and/or electricity which deleteriously altered skin and/or other tissue and acne disfiguration occasioned by oil build-up in blocked skin follicles.

26. The method of claim 22 where the applying is twice or three times daily by foam or spray until the infection subsides.
